(19) United States
(12) Patent Application Publication
(10) Pub. No.: US 2025/0391504 A1
(43) Pub. Date: Dec. 25, 2025

(54) DEVICE AND METHOD TO MONITOR GENETIC MUTATIONS

(71) Applicant: SOPHiA GENETICS S.A., Rolle (CH)

(72) Inventors: Slawomir KUBIK, Rolle (CH); Jonathan BIELER, Renens (CH); Yohann NEDELEC, Rolle (CH); Zhenyu Xu, Nyon (CH)

(73) Assignee: SOPHiA GENETICS S.A., Rolle (CH)

(21) Appl. No.: 19/249,610

(22) Filed: Jun. 25, 2025

Related U.S. Application Data
(60) Provisional application No. 63/664,162, filed on Jun. 25, 2024.
(60) Provisional application No. 63/693,704, filed on Sep. 11, 2024.

(51) Int. Cl. G16B 20/20; G16B 20/10; G16B 40/10; G16H 10/40; G16H 15/00; G16H 50/30; G16H 50/70

(52) U.S. Cl. CPC G16B 20/20; G16B 20/10; G16B 40/10; G16H 10/40; G16H 15/00; G16H 50/30; G16H 50/70

(57) ABSTRACT

A device and method for monitoring alleles in samples may involve obtaining lists of variants with support metrics from the samples, reconciling and combining the variant information into a single list with support metrics, transmitting the combined list to a dynamic user interface, selecting markers of interest based on user criteria from the interface, and generating an interpretation and displaying metrics based on user-defined parameters. The method may streamline the process of analyzing genetic data by consolidating variant information, facilitating user interaction through a dynamic interface, and providing customizable interpretation options for efficient and user-friendly allele monitoring.

### CROSS REFERENCE TO RELATED APPLICATIONS

The present application claims the benefit of U.S. Patent Application No. 63/693,704 for A COMPUTER-IMPLEMENTED METHOD TO MONITOR GENETIC MUTATIONS, filed Sep. 11, 2024; and U.S. Patent Application No. 63/664,162 for A DEVICE AND METHOD TO MONITOR GENETIC MUTATIONS filed Jun. 25, 2024, the entire contents of which are incorporated herein by reference.

### FIELD OF THE INVENTION

The present disclosure may be directed to a device composed of an analytical pipeline and a computer interface, to analyze genetic data from multiple samples, representing one patient over multiple time points or different subjects, sampled at the same or different time points. The system and method may be used to monitor the resurgence of genetic diseases, such as cancer, after a treatment. Because the system and method may be configured to allow a fully customizable analysis by the user, available clinical knowledge can be integrated in the assessment. The device may be designed to store, combine, and report when prompted by the user the full level of existing evidence, improving the information to support clinical decisions.

### BACKGROUND

Genetic analyses have entered many fields, including microbiology, crop sciences, environmental surveys, and medicine, with applications ranging from microbial monitoring, pre-natal testing, and identification of rare hereditary diseases, to diagnosing and monitoring of cancers. The advent of next-generation sequencing (NGS) allowed the generation of an ever-growing amount of genetic data for a constantly decreasing cost, supporting the widespread adoption of genetic assays. Analytical tools, however, need to be developed to help practitioners who are not bioinformatics experts access and harness the technology. Existing tools generally focus on the analysis of a given sample, with insights ranging from the identification of genetic variants present within the sample to the annotation of these variants. Some applications, however, require comparing multiple samples, which can represent multiple sampling times, individuals, or sampling sites. Supporting the widespread adoption of such applications by non-experts requires methods that allow the end users to interpret jointly the results of multiple genetic assays while setting the parameters based on their knowledge of the studied cases.

In a clinical context for example, repeated genetic analyses can improve diagnosis and prognosis for some diseases such as cancer. As a non-limiting example, Acute Myeloid Leukemia (AML) is a heterogeneous clonal disease caused by abnormal proliferation of blood cells of the myeloid lineage. Despite recent advances in supportive care and targeted therapy, relapses frequently occur, which are potentially associated with drug resistance, and often lead to poor outcomes for the relapsing patient.

During diagnosis, stratification of patients can be done based on rearrangements (translocations, deletions, and copy number alterations) identified by cytogenetic analysis. Furthermore, for accurate prognosis and refining the treatment options, it is crucial to identify the exact molecular changes (i.e., pathogenic mutations) of a patient.

Measurable Residual Disease (MRD) is one of the characteristics associated with the clinical outcome of diseases such as AML and was shown to be a valuable prognostic factor. Currently, MRD is often used after intensive chemotherapy as a prognostic factor to help stratify patients, to select the most appropriate consolidation therapy (e.g., in post-remission treatment for intermediate-risk patients, MRD positive patients receive allogeneic stem cell transplantation and MRD negative receive autologous stem cell transplantation), but emerging uses for MRD data include: selection of the type of allogeneic stem cell transplantation therapy (donor, conditioning), monitoring after stem cell transplantation (to allow intervention), and determining drug efficacy as a surrogate endpoint in clinical trials.

NGS-based methods can assess and quantify multiple mutations simultaneously and should be applicable to as many as 90% of AML patients. Contrary to PCR-based methods, NGS provides the ability to detect variants in multiple genes using a single assay without the need to design and validate multiple mutation specific assays. This allows one to discover emerging mutations during the course of monitoring. NGS-based assays can achieve limit of detection similar to PCR-based methods. NGS assays for MRD can target genomic regions identified at diagnosis or use a mutation-agnostic panel. If an agnostic panel approach is used, emerging variants not found at diagnosis should be reported only if confidently detected above background noise.

The analysis of NGS data necessitates dedicated tools and expertise, and the detection of low-frequency variants such as those characteristics of MRD is especially challenging. State-of-the-art reagents, algorithms, and bioinformatic pipelines to reconcile and analyze multiple genetic datasets and support clinical assessment must be integrated to support the transfer of research knowledge to clinical researchers. The resulting devices must, however, let the users dictate the detailed parametrization of the analyses, to ensure that expert knowledge can be integrated in the assessment.

While dynamic user interfaces exist to track specific types of genetic variants, in the context of diversity of the immune repertoire (e.g., ARResT/Interrogate), none exist in the MRD context. Instead, in the context of MRD and temporal mutation tracking, current solutions create automatic reports with non-customizable charts or graphs using pre-built macros in Microsoft Excel® (e.g., LymphoTrack), or allow users to regenerate time series graphs after modifying choices of markers and plotting options (e.g., Ion Reporter Software). However, such conventional approaches include interfaces that are not dynamic and/or not fully customizable, and instead require regeneration of a new plot after changing the options. In addition, such conventional options lack many of the metrics described below (e.g., customizable thresholds for MRD positivity, MRD score based on levels of noise and signal).

Thus, there is a need for devices to analyze multiple datasets, such as those needed in the context of MRD. Such devices must provide a dynamic user interface allowing for full customization and must enable comprehensive reporting in terms of markers, support values, and overall metrics, such as the novel MRD score presented here, which considers levels of signals and noise over multiple user-selected markers as opposed to inferring the presence or absence of MRD solely based on the variant allele fraction (VAF) of the MRD markers or the tumor content inferred from the VAF or read coverages exceeding a threshold.

Additionally, one of the main challenges for MRD analysis, especially from cfDNA, or more generally, low frequency allele fractions, is that due to the low frequency allele fractions in the blood/other bodily fluids, the expected number of molecules of DNA harboring relevant mutations is extremely low (Venditti et al. 2019, GIMEMA AML 1310 trial of risk-adapted, MRD-directed therapy for young adults with newly diagnosed acute myeloid leukemia, Blood 134:935-945; Zviran et al. 2020, Genome-wide cell-free DNA mutational integration enables ultra-sensitive cancer monitoring, Nature Medicine 26:1114-1124; Short et al. 2025, Clinical use of measurable residual disease in adult ALL: recommendations from a panel of US experts, Blood Advances 9:1442-1451). Accordingly, there is a need for MRD solutions to be developed to detect MRD at very low frequency allele fractions.

Further to this point, the detection of MRD presents significant technical challenges, particularly when analyzing cfDNA samples where variant allele fractions (VAFs) are extremely low. Conventional approaches struggle to reliably detect the presence of cancer cells at very low concentrations, which may represent, as a nonlimiting example, only 0.01% to 0.0001% (10⁻⁴ to 10⁻⁶) of total cells in a sample. The present disclosure addresses these challenges through a novel approach that integrates molecular barcoding technology with sophisticated statistical analysis to enhance signal-to-noise discrimination. By calculating a collective MRD score that considers the signal-to-noise ratio across all user-selected markers, rather than relying solely on individual variant detection, the system achieves improved sensitivity for detecting residual disease. This approach enables reliable detection at VAF levels previously unattainable with conventional methods, providing clinicians with critical information for treatment decisions even when cancer cell presence approaches the theoretical limits of detection.

Although examples are provided throughout the disclosure wherein the provided systems and methods are utilized for MRD tracking and assessment, said systems and methods may be applied to any disease-informative metric capable of being assessed at various time points as well as for other applications requiring the joint analysis of multiple genetic data sets representing multiple time points, multiple subjects, and/or multiple sampling sites. The present system and method aim to produce a device to generate genetic information for patients suffering from, or suspected to suffer from AML or another condition, and to analyze the resulting data.

### SUMMARY

Aspects of the present disclosure relate to a computer-implemented method of monitoring alleles of at least one or more samples, the method comprising the steps of: (a) obtaining at least one or more lists of variants with support metrics, from the at least one or more sample, (b) extracting and reconciling information of the at least one or more input lists of variants and combining the information to produce a single combined list of variants with support metrics, (c) transmitting the single combined list of variants with support metrics of step (b), to a dynamic user interface, (d) selecting at least one or more markers of interest based on user selected criteria, from the dynamic user interface of step (c), and (c) creating an interpretation and displaying metrics from the set of parameters defined by the user.

Aspects of the present disclosure relate to a method, wherein the input lists of variants and support metrics are selected from one or more sources, at least one or more points of time, different subjects, and/or sampling sites.

Aspects of the present disclosure relate to a method, wherein the list of variants and support metrics are provided as a variant table in a vcf-format file.

Aspects of the present disclosure relate to a method, wherein the monitored genetic variants comprise single nucleotide variants, insertions or deletions, copy number variants, and/or structural variants.

Aspects of the present disclosure relate to a method, wherein the input lists of variants lists all positions with an alternative allele supporting in the at least one or more samples by at least one or more sequencing reads, with associated support values.

Aspects of the present disclosure relate to a method, wherein the combined list of variants lists all variants detected in the at least one or more samples, with associated support.

Aspects of the present disclosure relate to a method, wherein the associated support values comprise the number of reads, number of duplex sequences, and/or number of groups of reads potentially originating from the same molecule.

Aspects of the present disclosure relate to a method, wherein the monitored genetic variants comprise genomic signatures, inferred from more than one genomic position.

Aspects of the present disclosure relate to a method, wherein the monitored genomic signatures comprise tumor mutational burden, genomic instability, microsatellite instability, methylation patterns, and/or gene expression patterns.

Aspects of the present disclosure relate to a method, wherein the genomic signatures are inferred from input lists of variants.

Aspects of the present disclosure relate to a method, wherein the variants are annotated and the annotations comprise predicted pathogenicity, coding consequences, known disease association, and/or frequency in population.

Aspects of the present disclosure relate to a method, wherein the variant annotation is integrated in the lists of variants.

Aspects of the present disclosure relate to a method, wherein the dynamic user interface of step (c) displays the time at which the at least one or more sample was taken, the identifier of the given subject, and/or the annotation of variants.

Aspects of the present disclosure relate to a method, wherein the at least one list of variants is received from a genomic analysis platform via an application programming interface (API).

Aspects of the present disclosure relate to a method, wherein (i) all of the at least two input lists of variants are obtained via the same next generation sequencing (NGS) assay, (ii) the lists of variants comprise identified variants and associated support values, and/or (iii) the lists of variants comprise all positions with at least one or more reads supporting a reference allele, wherein each position with an alternative allele is supported by at least one read in one sample and the support values for the alternative alleles are reported for all samples.

Aspects of the present disclosure relate to a method, wherein the NGS assay comprises a whole-genome sequencing assay, a whole-exome sequencing assay, a comprehensive profiling assay, or a disease-specific assay.

Aspects of the present disclosure relate to a method, wherein the NGS assay is developed specifically for the subject based on a first identification of variants in said subject.

Aspects of the present disclosure relate to a method, wherein (i) each of at least two of the samples are obtained via different assays and/or the positions covered by all assays are considered, (ii) the lists of variants include identified variants and associated support values, and/or (iii) the lists of variants include all positions with at least one or more reads supporting a reference allele, wherein each position with an alternative allele is supported by at least one read in one sample and the support values for the alternative alleles are reported for all samples.

Aspects of the present disclosure relate to a method, wherein at least one of the different assays comprises a next-generation sequencing (NGS) assay.

Aspects of the present disclosure relate to a method, wherein the NGS assay comprises a whole-genome sequencing assay, a whole-exome sequencing assay, a comprehensive profiling assay, or a disease-specific assay.

Aspects of the present disclosure relate to a method, wherein the NGS assay comprises a whole-genome sequencing assay, a whole-exome sequencing assay, a comprehensive profiling assay, or a disease-specific assay, wherein the disease-specific assay is an acute myeloid leukemia (AML)-specific assay.

Aspects of the present disclosure relate to a method, wherein the NGS assay is developed specifically for the subject based on a first identification of variants in said subject.

Aspects of the present disclosure relate to a method, wherein the markers of interest are selected by the user based on (i) the support values extracted from sample-specific lists of variants, (ii) the frequency of the variant in at least one of the samples, (iii) prior knowledge, and/or (iv) the annotation of the variant, such as the predicted pathogenicity, coding consequences, known disease association, and/or frequency in population.

Aspects of the present disclosure relate to a method, wherein the user classifies variants as germline variants, clonal hematopoietic variants, or measurable residual disease variants based on (i) the frequency of the variant in at least one of the samples, (ii) prior knowledge, and/or (iii) the annotation of the variant, such as the predicted pathogenicity, coding consequences, known disease association, and/or frequency in population.

Aspects of the present disclosure relate to a method, wherein at least one metric is computed based on the number, proportion and/or support of user-selected markers.

Aspects of the present disclosure relate to a method wherein the at least one metric is reported in the dynamic user interface, stored in the device, and/or transmitted to another device.

Aspects of the present disclosure relate to a method wherein the at least one metric is a measurable-residual disease (MRD) score computed based on the comparison of expected rate of errors across the selected markers and the observed support across the selected markers.

Aspects of the present disclosure relate to a method, wherein the metric is a measurable-residual disease (MRD) score based on the number and/or proportion of user-selected markers exceeding a user-defined threshold of support.

Aspects of the present disclosure relate to a method, wherein the samples are used to investigate and/or monitor a somatic genetic disease.

Aspects of the present disclosure relate to a method, wherein the somatic genetic disease comprises cancer.

Aspects of the present disclosure relate to a method, wherein at least one of the input lists of variants is derived from a biopsy.

Aspects of the present disclosure relate to a method, wherein at least one of the input lists of variants is derived from a liquid biopsy.

Aspects of the present disclosure relate to a method, wherein at least one of the input lists of variants is derived from cell-free DNA (cfDNA).

Aspects of the present disclosure relate to a computer system for dynamically reporting to a user (i) clinical data of at least one or more samples, (ii) at least one or more samples databases including for at least each sample, a set of clinical parameters associated with clinical data for a given subject, and (iii) a set of display parameters for each diagnostic status, the computer system executing the steps of: (a) obtaining at least one or more input results as lists of variants (VT), from each sample, (b) extracting and reconciling information of the at least one or more lists of variants and combining the information to produce a single combined list of variants, (c) transmitting the single combined list of variants of step (b), to a dynamic user interface, (d) selecting at least one or more markers of interest based on user selected criteria, from the dynamic user interface of step (c), and (e) creating an interpretation and displaying support metrics from the set of parameters obtained from the dynamic user interface.

Aspects of the present disclosure relate to a computer system, wherein the input results originate from at least one or more sources, at least one or more points of time, one or more distinct subjects, and/or distinct sampling sites.

Aspects of the present disclosure relate to a computer system, wherein the list of variants is received as a variant table in a vcf-format file.

Aspects of the present disclosure relate to a computer system, wherein the list of variants comprises single nucleotide variants, insertions or deletions, copy number variants, and/or structural variants.

Aspects of the present disclosure relate to a computer system, wherein the list of variants comprises (i) the variants detected in the sample, with associated support, and/or (ii) all positions with an alternative allele supporting in the sample by at least one sequencing read, with associated support values.

Aspects of the present disclosure relate to a computer system, wherein the associated support values comprise the number of reads, number of duplex sequences, and/or number of groups of reads potentially originating from the same molecule.

Aspects of the present disclosure relate to a computer system, wherein the variants are annotated and the annotations comprise predicted pathogenicity, coding consequences, known disease association, and/or frequency in population.

Aspects of the present disclosure relate to a computer system, wherein the variants comprise genomic signatures, wherein the genomic signatures comprise genomic instability, tumor mutational burden, microsatellite instability, methylation profile, and/or gene expression profile.

Aspects of the present disclosure relate to a computer system, wherein the dynamic user interface of step (c) displays the time at which the at least one or more sample was taken, the identifier of the given subject, and/or variant annotation.

Aspects of the present disclosure relate to a computer system, wherein the list of variants is received from a genomic analysis platform via an application programming interface (API).

Aspects of the present disclosure relate to a computer system, wherein (i) all of the at least two input lists of variants are obtained via the same next generation sequencing (NGS) assay, (ii) the lists of variants comprise identified variants and associated support values, and/or (iii) the lists of variants comprise all positions with at least one or more reads supporting a reference allele, wherein each position with an alternative allele is supported by at least one read in one sample and the support values for the alternative alleles are reported for all samples.

Aspects of the present disclosure relate to a computer system, wherein the NGS assay comprises a whole-genome sequencing assay, a whole-exome sequencing assay, a comprehensive profiling assay, or a disease-specific assay.

Aspects of the present disclosure relate to a computer system, wherein the NGS assays is developed specifically for the subject based on a first identification of variants in said subject.

Aspects of the present disclosure relate to a computer system, wherein (i) at least two of the samples are obtained via different assays and the positions covered by all assays are considered, (ii) the lists of variants include the identified variants and associated support values, and/or (iii) the lists of variants include all positions with at least one or more read supporting a reference allele, wherein each position with an alternative allele is supported by at least one read in one sample and the support values for the alternative alleles are reported for all samples.

Aspects of the present disclosure relate to a computer system, wherein at least one of the different assays comprises a next-generation sequencing (NGS) assay.

Aspects of the present disclosure relate to a computer system, wherein the NGS assay comprises a whole-genome sequencing assay, a whole-exome sequencing assay, a comprehensive profiling assay, or a disease-specific assay.

Aspects of the present disclosure relate to a computer system, wherein the NGS assay is developed specifically for the subject based on a first identification of variants in said subject.

Aspects of the present disclosure relate to a computer system, wherein the user selected criteria for the selection of markers of interest of step (d) comprises (i) the support values extracted from sample-specific VT, (ii), the frequency of the variants in at least one of the samples, (iii) prior knowledge, and/or (iv) the annotation of the variants, such as predicted pathogenicity, coding consequences, known disease association, and/or frequency in population.

Aspects of the present disclosure relate to a computer system, wherein the user classifies variants as germline variants, clonal hematopoietic variants, or measurable residual disease variants, based on (i) the frequency of the variants in at least one of the samples, (ii) prior knowledge, and/or (iii) the annotation of the variants, such as predicted pathogenicity, coding consequences, known disease association, and/or frequency in population.

Aspects of the present disclosure relate to a computer system, wherein at least one metric is computed based on the number, proportion and/or support of user-selected markers.

Aspects of the present disclosure relate to a computer system, wherein the at least one metric is reported in the dynamic user interface, stored in the device, and/or transmitted to another device.

Aspects of the present disclosure relate to a computer system, wherein the metric is a measurable-residual disease (MRD) score computed based on the comparison of expected rate of errors across the selected markers and the observed support across the selected markers.

Aspects of the present disclosure relate to a computer system, wherein the metric is a measurable-residual disease (MRD) score based on the number and/or proportion of user-selected markers exceeding a user-defined threshold of support.

Aspects of the present disclosure relate to a computer system, wherein the interpretation based on user-selected markers, thresholds and parameters can be exported into one or more downloadable reports.

Aspects of the present disclosure relate to a computer system, wherein the interpretation based on user-selected markers, thresholds and parameters can be transmitted to another device.

Aspects of the present disclosure relate to a method to calculate a measurable-residual disease (MRD) score based on a selection of markers, the method includes the steps of: (a) computing, for each marker, a level of support expected due to technical noise in an absence of the marker based on a pre-computed error rate and a total number of reads and/or number of groups of reads potentially originating from the same molecule covering a marker position; (b) measuring, for each marker, the level of support as the number of reads and/or number of groups of reads potentially originating from the same molecule supporting a presence of the marker; (c) computing, for each marker, a probability that the measured support is due to technical noise; (d) combining the probabilities obtained for all markers to obtain a probability that all measurements are due to technical noise; and (e) reporting the combined probability from step (d) as the MRD score

Aspects of the present disclosure relate to a method, wherein the MRD score is reported as the logarithm of the probability from step (d).

Aspects of the present disclosure relate to a method, wherein the MRD score is rescaled.

Aspects of the present disclosure relate to a method, wherein the MRD score is transmitted to a device.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 illustrates an embodiment of an environment in which the systems and methods of the present disclosure may be practiced.

FIG. 2 illustrates an embodiment of a block diagram of an electronic device.

FIG. 3 illustrates an example schematic representation of a method for assessing various samples at multiple time points, different subjects, and/or different sampling sites.

FIG. 4 illustrates an example schematic representation of the system and method for assessing various samples at multiple time points, different subjects, and/or different sampling sites.

FIG. 5 illustrates an example schematic representation of the workflow to go from DNA extracted from a sample to sequencing reads after subjecting the sequencing library to sequencing.

FIG. 6 illustrates a block diagram of an embodiment of the software device architecture.

FIG. 7 illustrates a schematic representation of a user workflow in accordance with one embodiment of the method.

FIG. 8 illustrates a schematic representation of the bioinformatic pipeline to obtain a modified variant table from sequencing reads.

FIG. 9 illustrates a schematic representation of the dynamic computer interface

FIG. 10 illustrates an embodiment of the workflow on the described system.

FIG. 11 illustrates access to the MRD interface from a broader genomic platform.

FIG. 12 illustrates genetic analysis outputs uploaded in the interface, which may be selected by the user for inclusion in the multi-sample interpretation.

FIG. 13 illustrates an example of an interface for the user to input thresholds for the interpretation.

FIG. 14 illustrates a list of variants reported in at least one of the selected analyses.

FIG. 15 illustrates a tool for users to filter variants based on criteria of their choice.

FIG. 16 illustrates a user-selected classification of variants as MRD markers.

FIG. 17 illustrates an example of interpretation.

FIG. 18 illustrates an example of interpretation.

FIG. 19 illustrates an example of a customized plot.

FIG. 20 illustrates a schematic representation of the functionality of the device of the present invention.

FIG. 21 illustrates a schematic representation of an exemplary DNA adaptor for use in DNA library generation.

FIG. 22 illustrates a schematic representation of an exemplary method of combining datasets for multiple samples and analyzing them jointly.

### DESCRIPTION

The particulars shown herein are by way of example and for purposes of illustrative discussion of the various embodiments only and are presented in the cause of providing what is believed to be the most useful and readily understood description of the principles and conceptual aspects of the methods and compositions described herein. In this regard, no attempt is made to show more detail than is necessary for a fundamental understanding, the description making apparent to those skilled in the art how the several forms may be embodied in practice.

The present disclosure will now be described by reference to more detailed embodiments. This present disclosure, however, may be embodied in different forms and should not be construed as limited to the embodiments set forth herein. Rather, these embodiments are provided so that this disclosure will be thorough and complete, and will fully convey the scope to those skilled in the art.

Unless otherwise defined, all technical and scientific terms used herein have the same meaning as commonly understood by those skilled in the art to which the present disclosure belongs. The terminology used in the description herein is for describing particular embodiments only and is not intended to be limiting. As used in the description and the appended claims, the singular forms ‘a,’ ‘an,’ and ‘the’ are intended to include the plural forms as well, unless the context clearly indicates otherwise.

Unless indicated to the contrary, the numerical parameters set forth in the following specification and attached claims are approximations that may vary depending upon the desired properties sought to be obtained and thus may be modified by the term about. At the very least, and not as an attempt to limit the application of the doctrine of equivalents to the scope of the claims, each numerical parameter should be construed in light of the number of significant digits and ordinary rounding approaches.

Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

### Definitions

The term “variant” or “genomic variant” refers to a difference in a genomic sequence relative to a designated reference sequence. In bioinformatics data processing, a variant is uniquely identified based on its chromosomal position (chr, pos) and the deviation from the reference genome at that position (ref, alt). Variants may encompass single nucleotide variants (SNVs), known as single nucleotide polymorphisms (SNPs) when referring to populations, insertions or deletions (INDELs), copy number variants (CNVs), and structural genomic modifications such as large-scale rearrangements, duplications, translocations, fusion, etc.

The term “mutation” or “mutated gene” refers to a gene in which at least one variant has been identified that was not present in a given reference sample or point. A “mutated gene status” may be classified as “mutated” in such instances. Otherwise, said status may be denoted as “normal.” Such a classification is commonly utilized as a biomarker in cancer diagnostics and prognostics.

A “germline variant” refers to a variant inherited from at least one parent that differs from the wild-type genomic sequence as recorded in a reference database and is present in the majority of normal cells of an individual.

A “somatic variant,” also referred to as a “somatic mutation” or “somatic alteration,” denotes a genomic alteration arising in one or more somatic cells of an individual, such as those found in a tumor. Somatic variants are restricted to a subset of the cells of the individual.

A “DNA fragment” refers to a short piece of DNA resulting from the fragmentation of high molecular weight DNA. Fragmentation may have occurred naturally in the sample organism, or may have been produced artificially from a DNA fragmenting method applied to a DNA sample, for instance by mechanical shearing, sonification, enzymatic fragmentation and other methods. After fragmentation, the DNA pieces may be end repaired to ensure that each molecule possesses blunt ends. To improve ligation efficiency, an adenine may be added to each of 3′ blunt ends of the fragmented DNA, enabling DNA fragments to be ligated to adaptors with complementary dT-overhangs.

An “adapter” or “adaptor” refers to a short double-stranded or partially double-stranded DNA molecule of around 10 to 100 nucleotides (base pairs) which has been designed to be ligated to a DNA fragment. An adapter may have blunt ends, sticky ends as a 3′ or a 5′ overhang, or a combination thereof. For example, to improve ligation efficiency, an adenine may be added to each of 3′ blunt ends of the fragmented DNA prior to adaptor ligation, and the adapter may have a thymidine overhang on 3′ end to base-pair with the adenine added to the 3′ end of the fragmented DNA. The adaptor may have a phosphorothioate bond before the terminal thymidine on 3′ end to prevent an exonuclease from trimming the thymidine, thus creating a blunt end when the end of the adaptor being ligated is double-stranded.

A “partially double stranded adaptor” refers to an adaptor including both a double-stranded region and a single stranded region. The double stranded region of the adaptor contains the ligation domain, whereas the single stranded region contains the priming sequences used for subsequent library amplification, barcoding and/or sequencing. The single stranded region can either be composed of two single stranded arms, a 5′ arm and a 3′ arm, as is the case for so-called Y-shape adaptors, or the single stranded region of partially double stranded adaptor can form a hairpin or a loop, as it is the case for the so-called U-shape adaptors. The term partially double stranded adaptor refers thus both to Y-shape and U-shape adaptors or a combination thereof.

The term “A-tailing” refers to an enzymatic method for adding an adenosine nucleotide to the 3′ end of a DNA molecule.

The term “amplification” refers to a polynucleotide amplification reaction to produce multiple polynucleotide sequences replicated from one or more parent sequences. Amplification may be produced by various methods, for instance a polymerase chain reaction (PCR), a linear polymerase chain reaction, a nucleic acid sequence-based amplification, rolling circle amplification, and other methods.

The term “hybridization capture” or “target enrichment” refers to a targeted next generation sequencing method that uses long, biotinylated oligonucleotide baits (probes) to hybridize to the regions of interest. The DNA-bait complexes are then isolated from the rest of the DNA, leading to the overrepresentation of the DNA fragments matching the baits.

The term “ligation” refers to the joining of separate double stranded DNA sequences. The latter DNA molecules may be blunt ended or may have compatible overhangs to facilitate their ligation. Ligation may be produced by various methods, for instance using a ligase enzyme, performing chemical ligation, and other methods.

The term “sequencing” refers to reading a sequence of nucleotides as a string. High throughput sequencing (HTS) or next-generation-sequencing (NGS) refers to real time sequencing of multiple sequences in parallel, typically between 50 and a few thousand base pairs. Exemplary NGS technologies include those from Illumina, Ion Torrent Systems, Oxford Nanopore Technologies, Complete Genomics, Pacific Biosciences, and others. Depending on the actual technology, NGS sequencing may require sample preparation with sequencing adaptors or primers to facilitate further sequencing steps, as well as amplification steps so that multiple instances of a single parent molecule are sequenced, for instance with PCR amplification prior to delivery to flow cell in the case of sequencing by synthesis.

A “molecular tag” or “molecular barcode” or “molecular code” or “molecular identifier” refers to a molecular arrangement such as a nucleic acid sequence which is fully and uniquely specified by its string of nucleotides.

“Read trimming” or “read pre-processing” refers, in a bioinformatics workflow, to the filtering out, in the sequencing reads, of a set of nucleotides at the start of the read sequence string, such as for instance the nucleotides corresponding to the adaptor sequences, to extract the real DNA fragment sequence to be analyzed.

“Aligning” or “alignment” or “aligner” refers to mapping and aligning base-by-base, in a bioinformatics workflow, the pre-processed sequencing reads to a reference genome sequence, depending on the application. While the reads are expected to come from specific genomic regions, a given proportion of reads may originate from other locations in the genome. Therefore, for the purposes of this disclosure, reads are typically mapped against the whole genome, after which only those aligned to the regions of interest are selected for further analysis, while others are discarded.

The term “cDNA” is also known as “complementary DNA” or “copy DNA” and refers to synthetic DNA that was reverse transcribed from RNA (e.g., messenger RNA, microRNA, etc.) through a reaction using the enzyme reverse transcriptase.

“Mitochondrial DNA” refers to the circular chromosome found inside the cellular organelles called mitochondria, rather than the nucleus.

The term “cfDNA” is also known as “circulating free DNA” or “cell free DNA” and refers to partially degraded DNA fragments released to body fluids such as blood, urine, cerebrospinal fluid, etc. In some embodiments, cfDNA derives from solid or liquid tumors.

A “DNA library” refers to a collection of DNA products or DNA-adaptor products to that are in a state compatible with a given next-generation sequencing platform.

A “nucleotide sequence” or a “polynucleotide sequence” refers to any polymer or oligomer of nucleotides such as cytosine (represented by the C letter in the sequence string), thymine (represented by the T letter in the sequence string), adenine (represented by the A letter in the sequence string), guanine (represented by the G letter in the sequence string) and uracil (represented by the U letter in the sequence string). It may be DNA or RNA, or a combination thereof. It may be found permanently or temporarily in a single-stranded or a double-stranded shape. Unless otherwise indicated, nucleic acids sequences are written left to right in 5′ to 3′ orientation.

A “primer sequence” refers to a nucleotide sequence of at least 5 nucleotides in length comprising a region of complementarity to a target DNA a part or all of which is to be elongated or amplified.

### Prior Art Problems Addressed

In an embodiment, the present method and device aim to analyze data for a subject suffering from cancer or another condition in a way that supports comparison of data obtained at different time points, toward an assessment of changes of allele frequencies, such as those indicative of MRD, based on marker choices and thresholds selected by the user based on their expertise. There is a long felt need to develop a dynamic user interface that tracks specific types of genetic variants in the context of MRD. Current solutions create automatic reports with non-customizable charts or graphs using pre-built macros, or allow users to generate time series graphs after modifying choices of markers and plotting options.

These approaches require regeneration of a new plot after changing each option and they lack many of the metrics that the present invention provides (e.g., thresholds for MRD positivity, generation of an MRD score, the use of a unique barcoding system for library DNA preparation, consideration of variant allele fractions jointly across multiple samples, as discussed in more detail below).

There is a need for MRD tracking and assessment devices to analyze multiple datasets, over multiple time points. As such, those devices must provide a fully customizable dynamic interface and must enable comprehensive reporting in terms of a variety of factors, e.g., markers, support values, metrics, etc.

The present disclosure provides such a device and software-implemented method to gain insights from the joint analysis of genetic results obtained from multiple samples, representing either multiple time points to track mutation frequency, multiple individuals, or multiple sampling sites using parameters driven by expert knowledge. The software back-end may reconcile and combine multiple variant tables into a joint dataset.

First, the disclosure may provide a novel bioinformatic tool configured to identify and record support metrics for all positions with an alternative allele in at least one sample. This innovation may allow the end user to track markers or their choice independently of the support level in a reference sample.

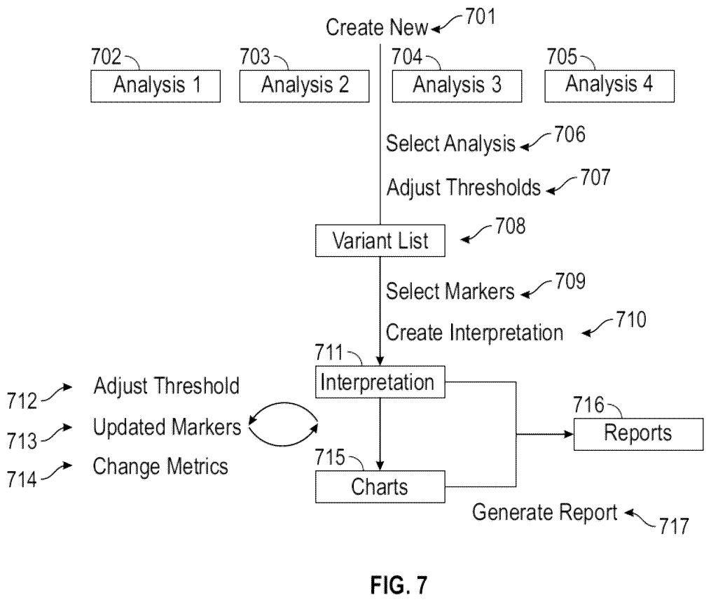

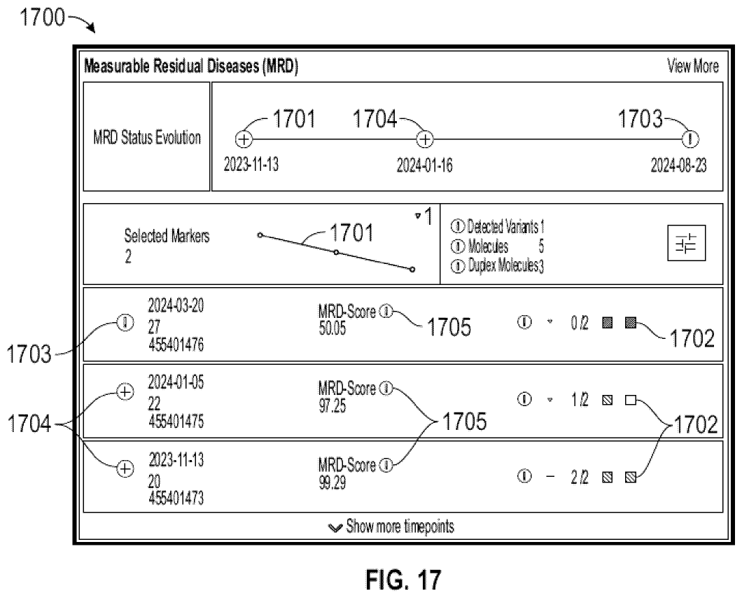

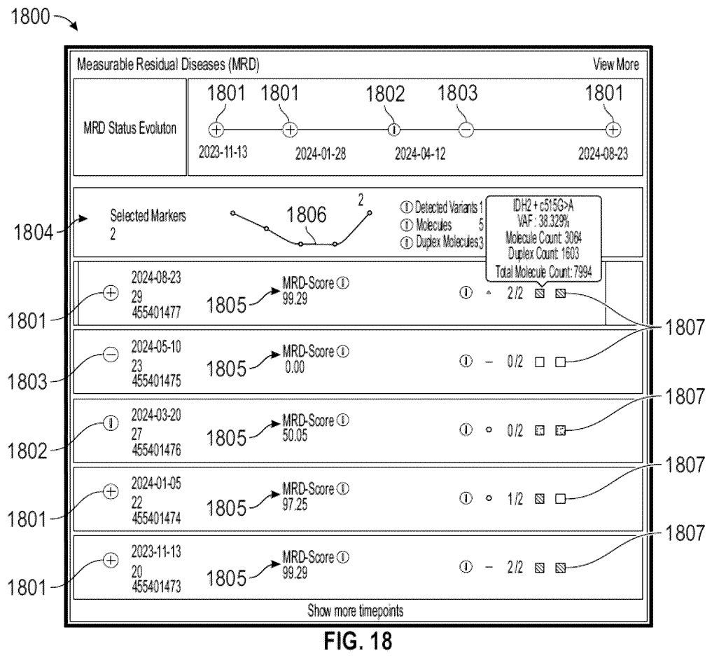

For example, as discussed in more detail below, and as illustrated in FIG. 7, FIG. 17, and FIG. 18, the present invention provides an opportunity for the user to select at least one marker to track wherein a comprehensive interpretation and report is generated regarding the MRD status.

Second, the disclosure may provide a dynamic user interface that can report information extracted from multiple samples, based on choices of markers, metrics, and thresholds selected by the user. The customizability and dynamism of the interface can be critical in ensuring that the user can use their expert knowledge to inform their assessment.

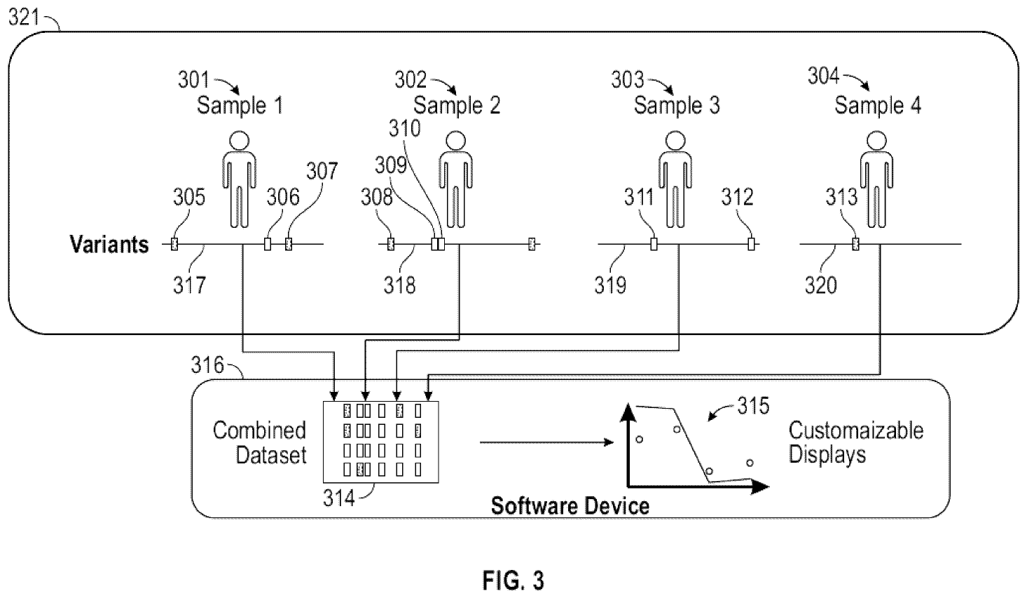

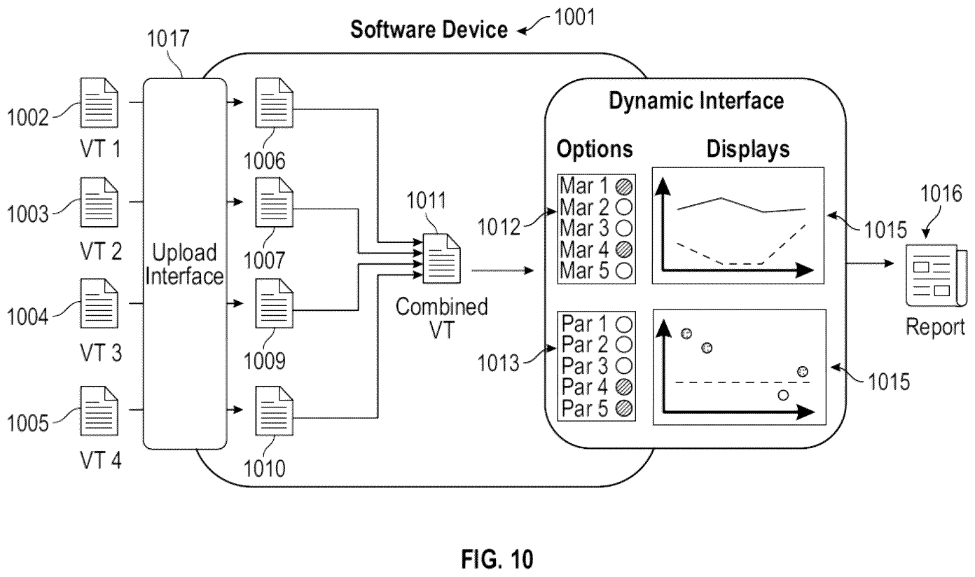

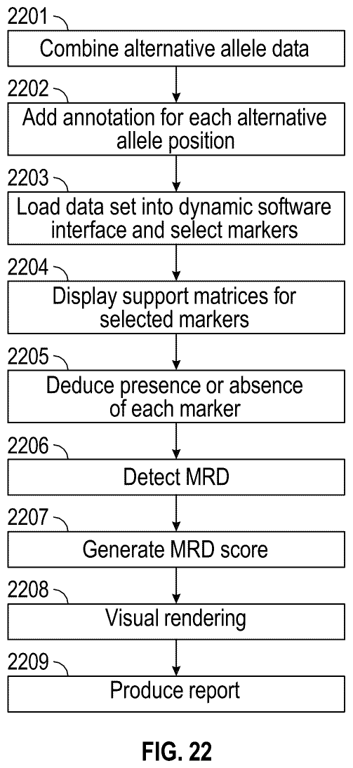

For example, as discussed in more detail below, and as illustrated in FIG. 3, FIG. 10, and FIG. 22, the dynamic user interface allows the user to select markers or interest, based on the criteria of their choice, display the support values for each marker and each sample, define support thresholds that are used to assess the presence of each marker, and generate comprehensive analyses and reports on the MRD status, all while being able to change each parameter at will.

Third, the disclosure may provide an MRD score computed based on a comparison of the total amounts of noise versus signal across markers selected by the user. The score may be recalculated after changes of the user choices, contributing further to ensuring that clinical knowledge can be integrated in the assessment.

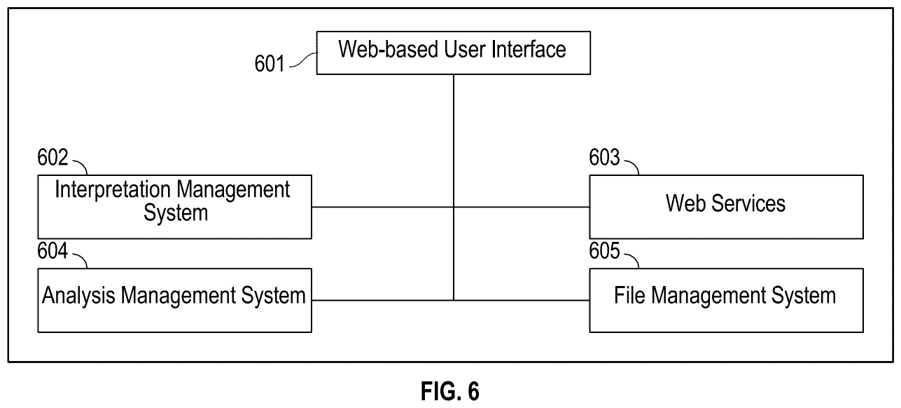

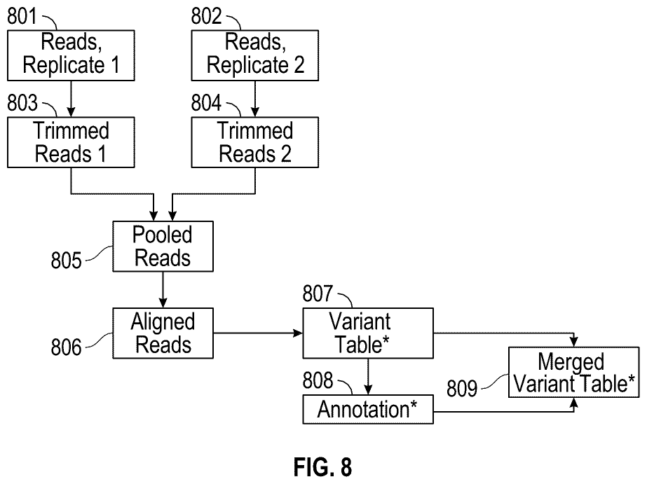

For example, as discussed in more detail below, and as illustrated by FIG. 3, FIG. 6, FIG. 8, FIG. 10, and FIG. 22, an MRD score can be generated based on completely customizable, user-defined parameters that are editable, and updateable. Furthermore, an MRD score can be generated from the combined datasets from the same sequencing assay, applied to multiple samples, which can represent multiple time points for one subject or different subjects.

The method and device may be used to monitor the resurgence of genetic diseases, such as cancer, after a treatment. In some embodiments, the method and device may be configured to allow a fully customizable analysis by the user and available clinical knowledge can be integrated in the assessment. The device may be designed to store, combine, and report, when prompted by the user, the full level of existing evidence, improving the information to support clinical decisions.

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized, and that structural changes and/or substitutions of various elements may be made without departing from the scope and spirit of this disclosure. The following detailed description is, therefore, not to be construed in a limited sense.

The present workflow addresses the limitations of conventional approaches by implementing a dynamic user interface that allows real-time updates and customization of data visualization without requiring regeneration of plots. This is achieved through a novel data structure that efficiently stores and retrieves combined variant information across multiple samples. The system utilizes a specialized caching mechanism that pre-computes and stores intermediate results, enabling rapid recalculation of metrics and instant updates to the visual representation when users modify parameters such as marker selection, thresholds, or plotting options. This technical implementation significantly reduces computational overhead and enhances user interaction, allowing for seamless exploration of complex genetic data in the context of MRD monitoring.

Furthermore, the workflow incorporates advanced algorithms for calculating novel metrics, such as the MRD score, which considers the total noise and signal across all user-selected markers. These calculations are performed in real-time using optimized mathematical models that efficiently process large volumes of genetic data. The system employs a modular architecture that separates data processing from visualization, allowing for independent scaling of computational resources as needed. This design enables the interface to handle whole-genome sequencing data while maintaining responsiveness, a feature not present in conventional solutions. Additionally, the implementation of customizable thresholds for MRD positivity is achieved through a flexible rule engine that dynamically applies user-defined criteria to the underlying data, providing a level of adaptability and precision in MRD assessment that was previously unavailable in existing tools.

The present workflow accommodates both uninformed and tumor-informed approaches to Measurable Residual Disease (MRD) monitoring, enhancing the flexibility and applicability of the system across various clinical scenarios. In the uninformed approach, the workflow begins with comprehensive sequencing of a tumor sample using methods such as whole-genome sequencing (WGS), whole-exome sequencing (WES), or analysis with a gene panel that covers known cancer-associated mutations. Concurrently, a normal sample may be sequenced using the same method to establish a baseline. This initial sequencing identifies tumor-specific mutations, which are then tracked in subsequent samples using the same sequencing approach after treatment or surgery. As such, the uninformed approach consists of repeatedly analyzing samples with the same panel, which is not selected based on the tumor profile. The present disclosure's dynamic interface and flexible data processing pipeline are designed to seamlessly integrate data from both tumor-informed and tumor-uninformed approaches to MRD monitoring. In tumor-uninformed workflows, the system can efficiently process and analyze data from repeated use of the same panel or sequencing method across multiple time points, while in tumor-informed scenarios, it can reconcile data from initial comprehensive sequencing and subsequent targeted panels. The workflow's ability to handle diverse input formats and its customizable marker selection feature allow users to effectively track variants across all time points, even when different sequencing methods are employed at different stages. This versatility enables the system to maintain analytical continuity and provide meaningful insights regardless of the chosen MRD monitoring strategy, whether it involves consistent use of a broad panel or a transition from a broad panel to more focused, tumor-specific assays.

The tumor-informed approach shares the initial steps with the uninformed method but diverges in the follow-up analysis. After identifying tumor-specific mutations, this approach involves developing a customized gene panel specifically targeting these mutations or selecting an existing gene panel that covers the detected mutations. This tailored panel may be implemented through various techniques, including multiplex amplicons (requiring primer development), capture methods (involving probe design), or even quantitative PCR for non-NGS tracking. The present invention's dynamic interface and flexible data processing pipeline are designed to seamlessly integrate data from both approaches. The system's ability to handle diverse input formats and its customizable marker selection feature allow users to effectively analyze and visualize MRD data regardless of the initial sequencing method or the subsequent targeted approach. This versatility enables clinicians and researchers to adapt their MRD monitoring strategy based on specific patient needs, tumor characteristics, or institutional preferences, while maintaining a consistent and intuitive analytical framework.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

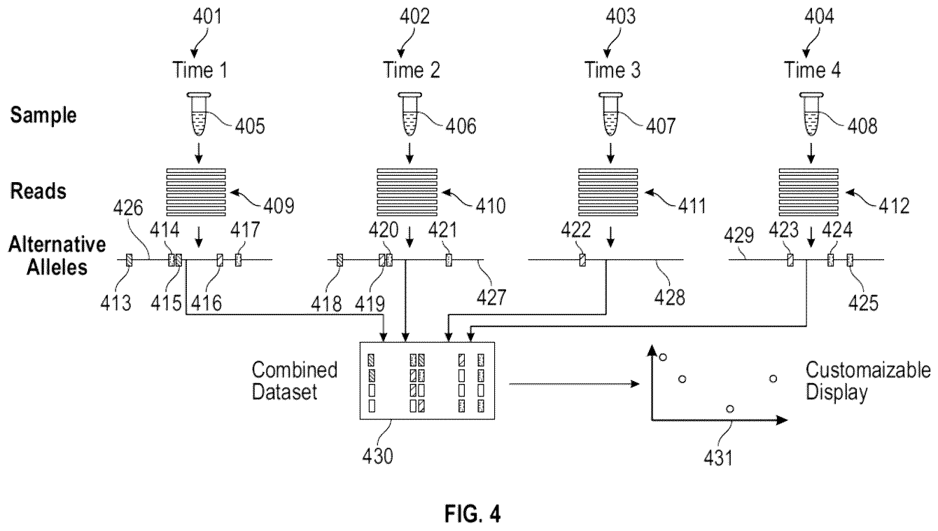

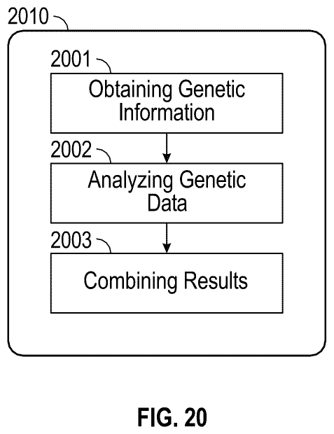

In an embodiment, the present disclosure describes, as illustrated by FIG. 20, a device 2010 that, referring to step 2001, obtains genetic information for genes relevant to acute myeloid leukemia (AML) or another condition. Referring to step 2002, the device may analyze the resulting genetic data to identify positions with alternative alleles, assess their support, and annotate them. Referring to step 2003, the device may combine results from different time points, individuals, and/or localizations in a dynamic computer interface to allow a user to evaluate changes of allelic frequencies, for example, to assess the presence of measurable residual disease (MRD), based on customizable marker choices, thresholds, and metrics (FIGS. 3 and 4).

Method of Extracting and Processing Nucleic Acids from a Sample

The method illustrated in FIG. 3 is a high-level schematic of one embodiment of the present disclosure. A software-implemented device 316 may receive as an input, the results from a genetic assay 321 (variants or positions with alternative alleles, with associated support values) obtained from multiple samples (Samples 1-4 301-304) which can correspond to multiple time points, different subjects, and/or different sampling sites. In the schematic illustrated in FIG. 3, the identified variants 305-313 for each sample are represented by rectangles along hypothetical reference genomic sequences 317-320.

As illustrated in FIG. 3, the variant information from the different samples 301-304 may be combined in a joint dataset 314 using a software device 316. Variants present in the reads produced for at least one of the time points but not in at least one of the others may be included in the combined dataset 314 with a support value of 0 for the samples in which they were not detected. As an example, variant 305 is present in the sample 1, in the sample 2 (as variant 308), but not in the samples 3 or 4. In the combined dataset 314, it therefore has a support value of 0 for samples 3 and 4. The joint dataset 314 may be received by a user interface and customizable displays 315 may be generated. In some embodiments, the user interface is a dynamic user interface and displays a pattern from the joint analysis of the different samples based on custom parameters.

The system and method illustrated in FIG. 4 is an exemplary schematic representation of one embodiment of the present disclosure. As illustrated in FIG. 4, for each time point 401-404, reagents may be used to produce, from DNA isolated from the samples 405-408, a sequencing library, which is then subject to high-throughput sequencing to produce sequencing reads 409-412. As illustrated in FIG. 4, for each time point 401-404, the sequencing reads 409-412 are analyzed and all alternative alleles 413-425 on the reference genome 426-429 are identified and recorded, together, with their support. In some embodiments, RNA is extracted from the samples and reverse transcribed to generate cDNA.

Non-limiting examples of reagents used to generate a sequencing library include enzyme-based fragmentation mixes, ligases, primers, adapters, kinases, deoxynucleotide triphosphate (dNTP), ATP, polymerase, buffers, ions (e.g., magnesium), reverse transcriptase, primers.

As illustrated in FIG. 4, the data 430 from different time points are combined and uploaded in a dynamic interface, which provides a display 431, which is an assessment of the evolution of markers across time points, using customizable markers and parameters. Variants present in the reads produced for at least one of the time points but not in at least one of the others are included in the combined dataset 430 with a support value of 0 for the samples in which they were not detected. As an example, variant 413 is present in the reads 409 (as variant 413) and 410 (as variant 418) from time points 1 and 2, respectively, but in the reads 411 and 412 from time points 3 and 4.

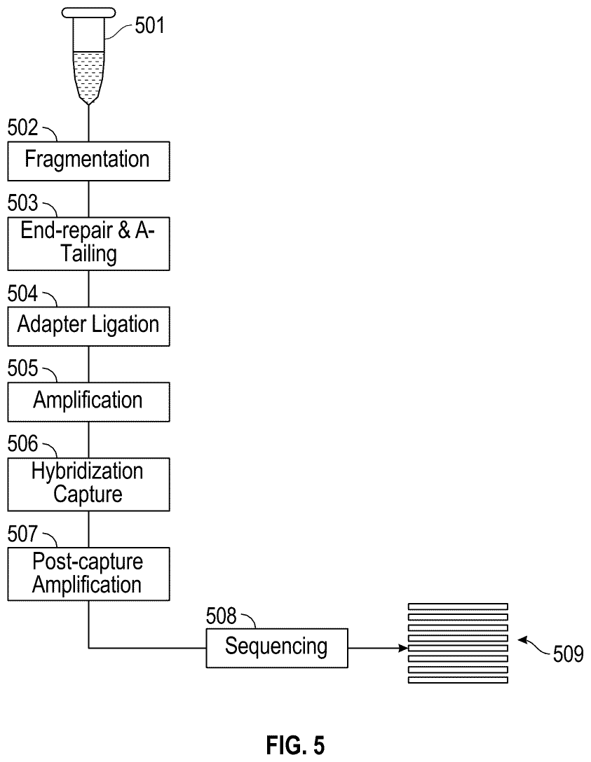

In some embodiments, a kit is used to obtain and analyze genetic data for at least one sample 501 from a subject suffering from, or suspected to suffer from, cancer (e.g., acute myeloid leukemia; AML) or another condition of interest. As illustrated in FIG. 5, the method of the present disclosure may include the following steps. Nucleic acid is isolated from the sample 501 of interest using a pre-existing DNA-isolation or RNA-isolation method.

DNA-isolation or DNA purification methods include, but are not limited to any known method to the skilled artisan, e.g., lysing extracted DNA from a sample using e.g., a detergent (e.g., sodium dodecyl sulphate, Triton X-100), separating the soluble DNA from the cell debris and other insoluble material, binding the DNA of interest to a purification matrix (e.g., silica), wash the bound DNA to remove impurities, and elute the bound DNA from the purification matrix. If needed, the isolated DNA is divided into multiple aliquots and each one is individually processed through steps 502-505.

RNA-isolation or RNA purification methods include, but are not limited to any known method to the skilled artisan, e.g., phenol-chloroform extraction (i.e., an extraction method that uses organic solvents to separate RNA based on the differential solubility of cellular components), column-based extraction and purification (i.e., using silica membranes or filters in a centrifuge to preferentially bind and elute RNA), magnetic bead-based extraction and purification (i.e., employing magnetic particles coated with RNA-binding surfaces to capture RNA from a solution). In some embodiments, after the RNA is extracted and purified, it is reverse transcribed into DNA (e.g., cDNA).

Referring to step 502, the DNA is optionally fragmented. Fragmentation 502 can be performed using any method known to the skilled artisan, including, but not limited to, mechanical shearing, sonication, ultrasonication, enzymatic fragmentation, partial digestion, restriction enzyme digestion.

Fragmentation 502 may result in a fragmented DNA being 50 to 10000 base-pairs in length. In some embodiments, fragmentation 502 may result in fragmented DNA being 50 base-pairs to 500 base-pairs in length. In some embodiments, fragmentation 502 may result in fragmented DNA being 500 to 1000 base-pairs in length. In some embodiments, fragmentation 502 may result in fragmented DNA being 1000-2000 base-pairs in length. In some embodiments, fragmentation 502 may result in fragmented DNA being 2000-3000 base-pairs in length. In some embodiments, fragmentation 502 may result in fragmented DNA being 3000-4000 base-pairs in length. In some embodiments, fragmentation 502 may result in fragmented DNA being 4000-5000 base-pairs in length. In some embodiments, fragmentation 502 may result in fragmented DNA being 5000-6000 base-pairs in length. In some embodiments, fragmentation 502 may result in fragmented DNA being 6000-7000 base-pairs in length. In some embodiments, fragmentation 502 may result in fragmented DNA being 7000-8000 base-pairs in length. In some embodiments, fragmentation 502 may result in fragmented DNA being 8000-9000 base-pairs in length. In some embodiments, fragmentation 502 may result in fragmented DNA being 9000-10000 base-pairs in length.

The DNA fragments may derive from cfDNA, cDNA, genomic DNA, or mitochondrial DNA and may be sized-fractionated, for example by agarose gel electrophoresis; gel chromatography; equilibrium density-gradient centrifugation, including sucrose gradient centrifugation, percol gradient centrifugation, cesium-chloride centrifugation; and other means known to the skilled artisan.

Referring to step 503, the DNA fragments can be processed through end-repair and A-tailing. After fragmentation 502, the extracted DNA can be end-repaired or end-polished and a single adenine base can be added to form an overhang by an A-tailing reaction. This A-overhang allows adapters containing a single thymine base to pair with the DNA fragments.

Referring to step 504, Y-shaped adaptors, containing primer-binding sites, are ligated to the ends of the DNA fragments. In some embodiments, the Y-shaped adapters include one of a diversity of molecular barcodes.

In some embodiments, the addition of molecular barcodes or molecular tags rely upon a unique tagging of each of the DNA fragments prior to amplification and sequencing, so that it is possible to group sequencing reads in families of reads associated with a specific tag. This facilitates the explicit detection and statistical consideration of errors introduced after the tagging, as it is unlikely that the very same error systematically repeats over all amplified and sequenced amplicon copies of the uniquely tagged parent DNA fragment.

In “Enhancing the accuracy of next-generation sequencing for detecting rare and subclonal mutations”, Nature Reviews Genetics, Vol. 18, pp. 269-285, May 2018 Salk et al. distinguishes between exogenous molecular barcodes as random or semi-random sequences which are artificially (physically) incorporated into either the PCR primers or the sequencing adaptors on the one hand, and endogenous molecular barcodes which may be identified as naturally (virtually) occurring fragmentation points (also known as shear points) at the ends of DNA molecules when preparing the DNA library using ligation.

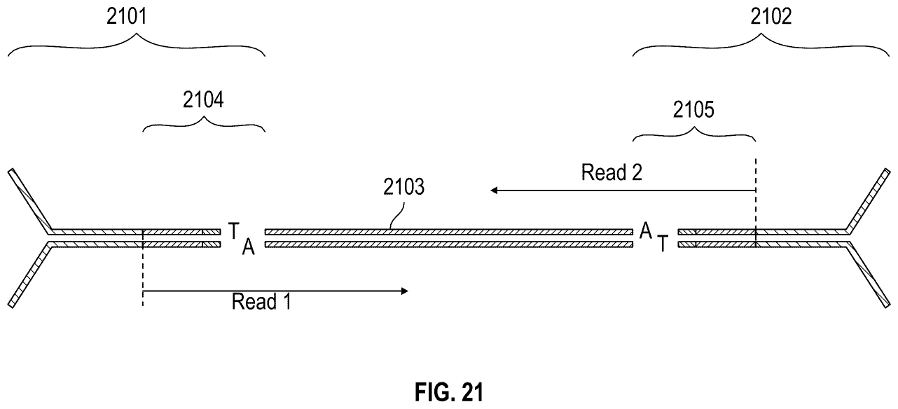

FIG. 21 shows an embodiment of the ligation step 504 of two adaptors 2101, 2102 to each end of a DNA fragment 2103. Each adaptor 2101, 2102 as shown in the exemplary embodiment as illustrated by FIG. 21 may comprise a partially double-stranded molecule of DNA with a single nucleotide (T) 3′ overhang at the end to be annealed to the double-stranded fragmented DNA. Each adaptor 2101, 2102 comprises a double stranded segment 2104, 2105 at one end which constitutes a spacer sequence (SS) separating the adaptor 2101, 2102 from the DNA fragment 2103 nucleotide sequences in subsequent high-throughput sequencing reads (Read 1, Read 2).

In a possible embodiment as illustrated by FIG. 21, the latter spacer sequence end may contain a single-nucleotide T 3′ overhang, but other embodiments are also possible as will be apparent to those skilled in the art, for instance it may be blunt ended or it may be substituted by another 3′ or 5′ overhang, so as to facilitate the ligation step 504 of the adaptor 2101, 2102 to the target double stranded DNA molecules 2103 (e.g., genomic DNA or gDNA).

An adaptor comprises a double-stranded sequence at the end being annealed to the double-stranded DNA. In this regard, one of the two strands of the double-stranded sequences of the adaptor will be ligated to the 3′ end of the fragmented double-stranded DNA, and the other of the two strands of the double-stranded sequences of the adaptor will be ligated to the 5′ end of the fragmented double-stranded DNA.

The ends of the double-stranded sequences of the adaptors being ligated to the fragmented double-stranded DNA are not limited and may comprise blunt ends, 3′ overhangs, and 5′ overhangs. In this regard, 5′ ends of the adaptors being ligated could either terminate with a 5′-phosphate or a 5′-OH. If a 5′-OH is at the adaptor end to be ligated to the target nucleic acid, it may be necessary to use a polynucleotide kinase to complete the backbone and join 5′-OH of the adapter to 3′-OH of the fragmented DNA.

Referring to step 505 of FIG. 5, DNA fragments with ligated adaptors are amplified using primers that incorporate primer-binding sites for subsequent sequencing-by-synthesis and sample-specific indexes. Amplification 505 can be performed using any known method, e.g., PCR.

In some embodiments, the result is a whole-genome sequencing library. In one embodiment, the whole-genome sequencing library is subjected to sequencing-by-synthesis 508, producing sequencing reads 509 spreads across the genome.

In one embodiment, part or all of the whole-genome sequencing library is used as input for hybridization capture, referring to step 506, using target-specific capture probes that match genomic regions of interest. DNA fragments and the hybridized matching probes are bound to streptavidin beads. The DNA-bead complexes are retrieved and cleaned up.

After release from the probes, referring to step 507, the DNA fragments are amplified using primers binding to the already incorporated primer-binding sites. In some embodiments, after clean-up, the amplified DNA fragments constitute the capture library. The capture library is subjected to sequencing-by-synthesis 508, producing sequencing reads 509 corresponding in majority to the targeted genomic regions.

In some embodiments, target genomic regions belong to genes associated with any disease or condition. In some embodiments, target genomic regions belong to any gene of interest.

In some embodiments, target genomic regions belong to genes associated with cancer. Cancer types can be grouped into broader categories. The main categories of cancer include: carcinoma (meaning a cancer that begins in the skin or in tissues that line or cover internal organs, and its subtypes, including adenocarcinoma, basal cell carcinoma, squamous cell carcinoma, and transitional cell carcinoma); sarcoma (meaning a cancer that begins in bone, cartilage, fat, muscle, blood vessels, or other connective or supportive tissue); leukemia (meaning a cancer that starts in blood-forming tissue (e.g., bone marrow) and causes large numbers of abnormal blood cells to be produced and enter the blood; lymphoma and myeloma (meaning cancers that begin in the cells of the immune system); and central nervous system cancers (meaning cancers that begin in the tissues of the brain and spinal cord).

Examples of carcinomas include, without limitation, giant and spindle cell carcinoma, small cell carcinoma, papillary carcinoma, squamous cell carcinoma, lymphoepithelial carcinoma, basal cell carcinoma, pilomatrix carcinoma, transitional cell carcinoma, papillary transitional cell carcinoma, an adenocarcinoma, a gastrinoma, a cholangiocarcinoma, a hepatocellular carcinoma, a combined hepatocellular carcinoma and cholangiocarcinoma, a trabecular adenocarcinoma, an adenoid cystic carcinoma, an adenocarcinoma in adenomatous polyp, an adenocarcinoma, familial polyposis coli, a solid carcinoma, a carcinoid tumor, a branchiolo-alveolar adenocarcinoma, a papillary adenocarcinoma, a chromophobe carcinoma, an acidophil carcinoma, an oxyphilic adenocarcinoma, a basophil carcinoma, a clear cell adenocarcinoma, a granular cell carcinoma, a follicular adenocarcinoma, a non-encapsulating sclerosing carcinoma, adrenal cortical carcinoma, an endometroid carcinoma, a skin appendage carcinoma, an apocrine adenocarcinoma, a sebaceous adenocarcinoma, a ceruminous adenocarcinoma, a mucoepidermoid carcinoma, a cystadenocarcinoma, a papillary cystadenocarcinoma, a papillary serous cystadenocarcinoma, a mucinous cystadenocarcinoma, a mucinous adenocarcinoma, a signet ring cell carcinoma, an infiltrating duct carcinoma, a medullary carcinoma, a lobular carcinoma, an inflammatory carcinoma, Paget's disease, a mammary acinar cell carcinoma, an adenosquamous carcinoma, an adenocarcinoma w/squamous metaplasia, a sertoli cell carcinoma, embryonal carcinoma, choriocarcinoma.

Examples of sarcomas include, without limitation, glomangiosarcoma, sarcoma, fibrosarcoma, myxosarcoma, liposarcoma, leiomyosarcoma, rhabdomyosarcoma, embryonal rhabdomyosarcoma, alveolar rhabdomyosarcoma, stromal sarcoma, carcinosarcoma, synovial sarcoma, hemangiosarcoma, kaposi's sarcoma, lymphangiosarcoma, osteosarcoma, juxtacortical osteosarcoma, chondrosarcoma, mesenchymal chondrosarcoma, giant cell tumor of bone, ewing's sarcoma, odontogenic tumor, malignant, ameloblastic odontosarcoma, ameloblastoma, malignant, ameloblastic fibrosarcoma, myeloid sarcoma, mast cell sarcoma.

Examples of leukemias include, without limitation, leukemia, lymphoid leukemia, plasma cell leukemia, erythroleukemia, lymphosarcoma cell leukemia, myeloid leukemia, basophilic leukemia, eosinophilic leukemia, monocytic leukemia, mast cell leukemia, megakaryoblastic leukemia, and hairy cell leukemia.

Examples of lymphomas and myelomas include, without limitation, malignant lymphoma, Hodgkin's disease, Hodgkin's, paragranuloma, malignant lymphoma, small lymphocytic, malignant lymphoma, large cell, diffuse, malignant lymphoma, follicular, mycosis fungoides, other specified non-Hodgkin lymphomas, myeloma, and multiple myeloma.

Examples of melanomas include, without limitation, malignant melanoma, amelanotic melanoma, superficial spreading melanoma, malignant melanoma in giant pigmented nevus, and epithelioid cell melanoma.

Examples of brain/spinal cord cancers include, without limitation, pincaloma, malignant, chordoma, glioma, malignant, ependymoma, astrocytoma, protoplasmic astrocytoma, fibrillary astrocytoma, astroblastoma, glioblastoma, oligodendroglioma, oligodendroblastoma, primitive neuroectodermal, cerebellar sarcoma, ganglioneuroblastoma, neuroblastoma, retinoblastoma, olfactory neurogenic tumor, meningioma, malignant, neurofibrosarcoma, neurilemmoma, malignant.

Examples of other cancers include, without limitation, a thymoma, an ovarian stromal tumor, a the coma, a granulosa cell tumor, an androblastoma, a leydig cell tumor, a lipid cell tumor, a paraganglioma, an extra-mammary paraganglioma, a pheochromocytoma, blue nevus, malignant, fibrous histiocytoma, malignant, mixed tumor, malignant, mullerian mixed tumor, nephroblastoma, hepatoblastoma, mesenchymoma, malignant, brenner tumor, malignant, phyllodes tumor, malignant, mesothelioma, malignant, dysgerminoma, teratoma, malignant, struma ovarii, malignant, mesonephroma, malignant, hemangioendothelioma, malignant, hemangiopericytoma, malignant, chondroblastoma, malignant, granular cell tumor, malignant, malignant histiocytosis, immunoproliferative small intestinal disease.

In some embodiments, target genomic regions belong to genes associated with autoimmune diseases, including but not limited to acquired hemophilia, acromegaly, agammaglobulinemia, alopecia areata, amyloidosis, ankylosing spondylitis, antiphospholipid syndrome, aplastic anemia, arteriosclerosis, Addison's disease, celiac disease, chagas disease, chronic autoimmune urticaria, Churg-Strauss syndrome, Cogan's disease, Crohn's disease, dermatitis herpetiformis, discoid lupus, eczema, endometriosis, eosinophilic esophagitis, eosinophilic fasciitis, Evans syndrome, giant cell myocarditis, giant cell arteritis, Graves' disease, Guillian-Barre syndrome, Hashimoto's thyroiditis, interstitial cystitis, Kawasaki disease, lupus, Lyme disease, mixed connective tissue disease, multiple sclerosis, narcolepsy, palindromic rheumatism, polymyalgia rheumatica, polymyositis, primary biliary cirrhosis, psoriasis, psoriatic arthritis, Raynaud's syndrome, reactive arthritis, rheumatic fever, rheumatoid arthritis, sarcoidosis, scleritis, Sjogren's syndrome, small fiber sensory neuropathy, Takayasu arthritis, testicular autoimmunity, type 1 diabetes, ulcerative colitis, undifferentiated connective tissue disease, and vitiligo.

In some embodiments, target genomic regions belong to genes associated with cardiovascular disease, including, but not limited to, heart failure, peripheral artery disease, coronary artery disease, arrhythmia, hypertension, congenital heart disease, cardiomyopathy, valvular heart disease, aortic aneurysm, deep vein thrombosis, pulmonary embolism, myocarditis, pericarditis, and rheumatic heart disease.

In some embodiments, target genomic regions belong to genes associated with neurological disease including, but not limited to, Parkinson's disease, spinal cord injury, stroke, Alzheimer's disease, amyotrophic lateral sclerosis, multiple sclerosis, epilepsy, migraine, Huntington's disease, peripheral neuropathy, traumatic brain injury, cerebral palsy, autism spectrum disorder, Tourette syndrome, dementia, meningitis, and neurofibromatosis.

In some embodiments, target genomic regions belong to genes associated with other diseases or conditions, including but not limited to, liver failure, kidney disease, sickle cell anemia, beta-thalassemia, muscular dystrophy, muscular atrophy, progeria, Wilson disease, Gaucher disease, Pompe disease, Rett syndrome, Ehlers-Danlos syndrome, Marfan syndrome, idiopathic pulmonary fibrosis (IPF), and amyloidosis.

In one embodiment, target genomic regions belong to the genes associated with AML. In some embodiments the target genomic regions belonging to genes associated with AML comprise CALR, CEBPA, DDX41, ETV6, EZH2, FLT3, IDH1, IDH2, JAK2, KIT, KRAS, MPL, NPM1, NRAS, PTPN11, RAD21, RUNX1, SF3B1, SRSF2, STAG2, TP53, U2AF1, and WT1. In other embodiments, other genomic regions are targeted.

In some embodiments, if molecular barcodes were used, the sequencing reads 509 potentially originating from the same molecule are identified based on their molecular barcodes. Adaptors, and barcodes where relevant, are trimmed from the reads. If multiple libraries were produced from aliquots of a given sample and sequenced, the sequencing reads 509 originating from the different aliquots are pooled. The trimmed and pooled reads are aligned to a reference genome.

Differences between the aligned reads and the reference genome are identified, including single-nucleotide variants (SNVs), insertion-deletions (indels), and fusion events. In some embodiments, for each site where an alternative allele is supported by at least one read 509, the number of reads, groups of reads potentially originating from the same molecule and duplex sequences supporting the reference and alternative alleles are recorded.

In some embodiments, the alternative alleles are annotated, using approaches that can include, but are not limited to, cross-referencing with existing knowledge databases and the use of tools to predict variant effects, such as the effect on the encoded protein or the pathogenicity for the subject. The results to report are selected by the user, and a report is produced.

A device may be presented to obtain genetic data and analyze the obtained data for a subject suffering from, or suspected to suffer from, cancer or another condition of interest. In an embodiment, the device includes a software to analyze and report the results of said sample. In an embodiment, the device includes a dynamic interface to combine results from multiple samples collected longitudinally as a time series and allow a user to produce reports of the time series based on custom parameters. In another embodiment, the device includes a kit for the preparation of one sample and a software to analyze and report the results of said sample.

In one aspect of the invention, a method for analyzing genetic data from subjects with or suspected of having acute myeloid leukemia (AML) or other conditions is provided. The method begins with sample collection, which may be a blood sample or a formalin-fixed paraffin-embedded (FFPE) sample. DNA or RNA is isolated from the sample using established DNA/RNA-isolation techniques. If necessary, the isolated DNA/RNA may be divided into multiple aliquots for individual processing through subsequent steps.

The method may continue with DNA fragmentation, end-repair, and A-tailing. Y-shaped adaptors, which contain primer-binding sites and unique molecular barcodes, are then ligated to the ends of the DNA fragments. These adapted DNA fragments are amplified using primers that incorporate additional primer-binding sites for subsequent sequencing-by-synthesis and sample-specific indexes, resulting in a whole-genome sequencing library.

In some embodiments, the whole-genome sequencing library, or a portion thereof, is used as input for a capture step. This step employs target-specific capture probes that correspond to regions of interest. In one implementation, the target regions may include genes such as CALR, CEBPA, DDX41, ETV6, EZH2, FLT3, IDH1, IDH2, JAK2, KIT, KRAS, MPL, NPM1, NRAS, PTPN11, RAD21, RUNX1, SF3B1, SRSF2, STAG2, TP53, U2AF1, and WT1. However, the invention is not limited to these specific genes, and other embodiments may target different sets of genes or genomic regions. The capture process involves binding DNA fragments and hybridized matching probes to streptavidin beads. These DNA-bead complexes are then extracted, cleaned, and the captured DNA fragments are amplified using primers that bind to the previously incorporated primer-binding sites. After cleanup, these amplified DNA fragments form the capture library.

The capture library is then subjected to sequencing-by-synthesis, generating sequencing reads that predominantly correspond to the targeted genomic regions. Following sequencing, the reads potentially originating from the same molecule are identified based on their molecular barcodes. Adaptors and barcodes are trimmed from the reads, and if the same sample was sequenced in different runs, reads from these different runs are pooled. The trimmed and pooled reads are then aligned to a reference genome.

A key feature of the invention is its comprehensive approach to variant identification. Differences between the aligned reads and the reference genome are identified, and importantly, for each site where an alternative allele is supported by at least one read, the method records the number of reads, groups of reads potentially originating from the same molecule, and duplex sequences supporting both the reference and alternative alleles. This approach differs from conventional methods that typically report only called variants. By reporting all alternative alleles, the present invention provides a more complete dataset for subsequent multi-time-point analysis and allows users to customize thresholds in MRD analysis based on the full spectrum of evidence.

The list of alternative alleles is then presented in a software interface, along with annotation information about each alternative allele retrieved from various databases. The pathogenicity of each alternative allele is predicted using pre-existing tools. The interface also provides users with the opportunity to add additional information before generating reports, enhancing the flexibility and customization of the analysis process.

Computer-Implemented Method to Identify Alternative Alleles and their Support

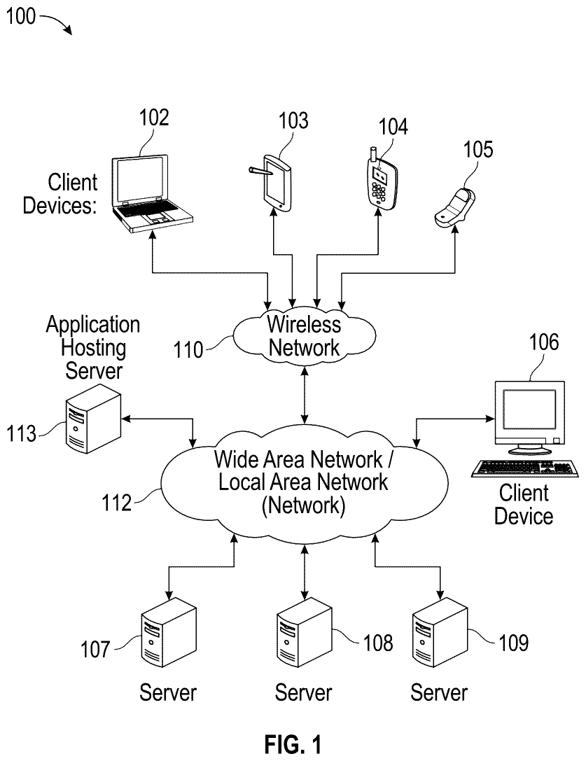

FIG. 1 illustrates components of one embodiment of an environment in which the present disclosure may be practiced. Not all of the components may be required to practice the present disclosure, and variations in the arrangement and type of the components may be made without departing from the spirit or scope of the present disclosure. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. The client devices 102-106 may include, for example, at least one of desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 1 also illustrates application hosting server 113.

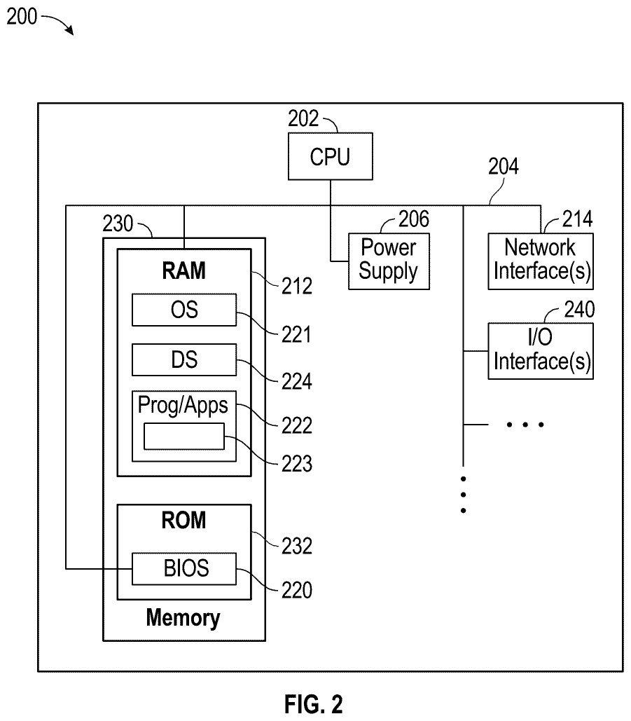

FIG. 2 illustrates a block diagram of an electronic device 200 that can implement one or more aspects of an apparatus, system, and method for measurement and secure transmission of physical properties (the “Engine”) according to one embodiment of the present disclosure. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software, and other tools necessary to implement or facilitate methods and systems according to embodiments of the present disclosure. The elements may exist on a single computer or be distributed among multiple computers, servers, devices, or entities.

The power supply 206 contains one or more power components and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can case processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system, and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone, or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as IOS, Android, Windows Mobile, and the like.

Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Arca Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPV6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present disclosure include apparatuses, systems, and methods implementing the Engine. Embodiments of the present disclosure may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

The method may be carried out by a software device located on a cloud computing server to permit decentralized analysis. In an embodiment, the cloud computing server may comprise a global center that provides central services, such as user authentication and authorization. In one embodiment, the cloud computing server may comprise at least one regional center to provide file management, storage, and other functionalities. It is contemplated that this permits the users to access the software from a server that complies with local requirements and regulations.

A web-based user interface 601 module and its dedicated services 107-109 is illustrated in FIG. 6. As illustrated, the user interface 601 may be coupled to an interpretation management system 602, which is responsible for generating interpretations that can include a MRD status, an analysis management system 604, a web service 603, and a file management system 605. In an embodiment, the analysis management system 604 may be responsible for data storage and analysis (i.e., bioinformatic pipelines) and the triggering or storing thereof. In an embodiment, the interpretation management system 602 may be responsible for generation and interaction with the frontend, for example, assessing inputs from the end user.

In one embodiment, the user interface may be accessible via a browser. Of course, in other embodiments, the user interface 601 may be accessible via a downloadable application, such as a mobile application. The user interface 601 may provide access to an interpretation module, for example the interpretation management system 602, which can manage the sorting, filtering, and selecting of sets of merged variants. It is contemplated that the different components of the device can utilize well-defined application programming interfaces (APIs) to help integration with other systems.

In one embodiment, the file management system 605 may be a digital infrastructure adapted for organizing, storing, and enabling retrieval of datasets of information, such as variant information, patient-specific records, and/or historical results (e.g., results of previous iterations of a given analysis or assessment, such as the first instance of an MRD score calculation for a given patient or the first instance of data collection from a given patient in a time series). The file management system 605 may provide secure access of such information to the analysis management system 604 or other components as demonstrated in FIG. 6.

In some embodiments, the device may utilize a subset of services 107-109 from pre-existing genomic analysis platforms, for example, SOPHIA DDM™. As a nonlimiting example, the device may comprise a dedicated user interface module and a dedicated service to calculate the MRD score. In such a nonlimiting example, the interface is provided as an add-on to the SOPHIA DDM™ platform and can receive datasets generated with any solution. As a non-limiting example, the datasets may have been generated with the SOPHIA DDM™ Residual Acute Myeloid solution that includes capture probes that target 23 genes linked to acute myeloid leukemia (AML) and treated with a bioinformatic pipeline, for example, the bioinformatics pipeline as illustrated in FIG. 8, that reports support values for all position with at least one read supporting an alternative allele in one sample.

In an embodiment, the MRD workflow described herein is implemented as an integrated component of a larger genomic analysis platform, sharing server resources and backend elements for efficient data processing and retrieval. The dynamic interface, while functioning as an add-on, may be seamlessly integrated with the platform's architecture, allowing direct data exchange and leveraging shared computational resources. This integrated approach enhances the system's ability to handle large volumes of genetic data, enabling real-time updates to the visual representation when users modify parameters such as marker selection, thresholds, or plotting options, while maintaining the flexibility for the interface to be deployed as a stand-alone software in alternative implementations.

In some embodiments, the device to dynamically analyze genetic results from multiple datasets in implemented in the Oncoportal™ Mutation Tracker add-on, provided with the SOPHIA DDM™ platform.

In some embodiments, the device may be independent from pre-existing genomic analysis platforms (e.g., SOPHIA DDM™) by duplication of the shared services. For example, the device can use a dedicated series of tools to absorb variants tables in diverse formats.

FIG. 7 illustrates a schematic representation of a user workflow in accordance with one embodiment of the method.

The method disclosed herein may correspond to a session and may be initiated by the creation of a new session 701. Upon creation of the new session 701, a plurality of genetic analysis outputs 702-705 may be listed. In one embodiment, each of the plurality of genetic analysis outputs 702-705 may correspond to a variant table, however, other forms of providing the genetic analysis outputs 702-705 are contemplated. In some embodiments, the genetic analysis outputs 702-705 include annotation information, such as pathogenicity predictions, position relative to genes, frequency in a population of interest, presence in clinical databases, association to drugs, presence in scientific literature, splicing effects, etc. The plurality of genetic analysis outputs 702-705 may be provided by a genomic platform linked to the device and/or from files uploaded by the user.

Referring to step 706 of FIG. 7, a user may select at least one of the plurality of genetic analysis outputs 702-705 to include in the interpretation analysis. Further, in some embodiments, the user may adjust the thresholds 707 for variant identification. Upon selection and/or adjustment, a list of variants 708 in the genetic analysis outputs 702-705 selected for interpretation analysis may be generated.

Referring to step 709 of FIG. 7, a user may select at least one marker to track and an interpretation 711 may be created, referring to step 710 of FIG. 7. In one embodiment, the interpretation 711 may comprise metrics summarizing the quality of the data, the signal present in the data, and metrics of interest to the user. As a non-limiting example, the metrics may include the number of markers with support above the selected thresholds in each sample. As another non-limiting example, the metrics may include a MRD score, calculated across samples based on the selected thresholds and markers. In one embodiment, the interpretation 711 may comprise at least one chart 715. In some embodiments, charts may be based on thresholds and parameters (e.g., markers) selected by the user. For example, a user could select the number of markers with support above a user-selected threshold in each sample, or an MRD score to display in the charts. In another example, a user could plot the number of positions with reads supporting a variant across samples. Of course, other means of displaying the interpretations are contemplated. The user can modify any of the at least one marker, threshold, and/or metric to display. Upon modification, the interpretations may be updated 712-714 and a new chart 715 may be displayed.

Referring to step 717 of FIG. 7, interpretations 711 and/or the at least one chart 715 may be exported into a report 716. In some embodiments, multiple, user-defined, charts may be exported into a report. In some embodiments, the report 716 may be a downloadable report. In an embodiment, the report 716 may be transmitted to another device and/or saved in the system. In another embodiment, overall metrics generated in the interpretation 711 may be transmitted to another device independently of the report 716. In yet another embodiment, the report 716 may be incorporated in a different report (e.g., a full medical report to be transmitted to the oncologist) or may be transmitted into an electronic health record (EHR) or EHR system.

In one embodiment, the device may include an analytical pipeline to identify, from sequencing reads, positions with potential genetic variants using various algorithms. The pipeline may clean sequencing reads and align them to a reference genome. Algorithms may be used to identify, based on the molecular barcodes, sequencing reads that potentially originate from the same molecule in the starting DNA material contained in the sample. The use of molecular barcodes may permit for grouping reads that likely came from the same starting molecule, but there may always some level of ambiguity due to factors like, for example, sequencing errors in the barcodes or chance duplications of barcodes. Therefore, groups of reads with matching barcodes are considered to “potentially” originate from the same molecule, rather than stating this with absolute certainty. This phrasing may acknowledge the probabilistic nature of associating sequencing reads with their source molecules, while still allowing the system to use this grouping information as a valuable support metric for variant detection and quantification.

In such embodiments, bioinformatic algorithms may be used to identify, from the aligned reads, positions where some of the reads of the sample present alternative alleles compared to the reference genome. The support for the alternative allele may then be calculated for each such position. Various tools and algorithms may then be used to annotate the alternative alleles, including predictions of pathogenicity, position with respect to genes, coding consequences, splicing effects, frequency in reference populations, citation in clinical databases, and the like.

In some embodiments, the system and method further comprises a bioinformatic pipeline configured to combine variant information and the associated support values inferred from genetic data obtained from the multiple samples. In one embodiment, the bioinformatic pipeline may further combine an annotation of variants with the variant information and the associated support values.

As illustrated in the representative schematic of a bioinformatic pipeline in FIG. 8, a modified variant table 809 from sequencing reads may be obtained. In the represented scenario, one sample is processed as two replicates 801-802. The two replicates are then trimmed into trimmed reads 803-804. The trimmed reads 803-804 are pooled into pooled reads 805. The pooled reads 805 are then aligned 806 to generate a variant table 807. Annotations 808 are generated from the variant table 807 and the variant table 807 and the annotations 808 are combined to generate a merged variant table 809. The asterisks indicate that all positions with some reads supporting the alternative allele are reported.

In various embodiments, the device may be compatible with any NGS assay, for example, target-enrichment panels, metagenomic sequencing, whole transcriptome sequencing, targeting methyl sequencing, whole exome sequencing, amplicon sequencing, anchored multiple PCR chemistry, RNA sequencing and whole-genome sequencing.

### Dynamic Computer Interface to Evaluate Measurable Residual Disease (MRD)

The present disclosure may provide a bioinformatic pipeline, e.g., as illustrated in FIG. 8, to combine alternative alleles or variants and the associated support values identified from genetic data obtained from different time points, different subjects, and/or different sampling sites, as illustrated in FIG. 3. In an embodiment, the method further provides a dynamic user interface, where the combined dataset 314 can be loaded and assessed by the user. The user can select markers of interest, based on criteria of their choice, and then display the support values for the markers of interest, for each of the samples. In one embodiment, user-defined support thresholds can then be used to assess the presence of each selected marker from each time point, subject, and/or sampling site. Metrics assessing the number of variants present in the samples can be computed for the selected variants.

For example, an MRD status can then be inferred for each sample if the number of markers above the user-selected threshold exceeds another user-selected threshold. In addition, a global MRD score may be calculated based on the total amount of noise and signal at the user-selected markers. The metric values and information displayed on the user interface can be updated in real time in response to changes made to any of the parameters (e.g., markers, thresholds, criteria, etc.). Selected metrics and displays can be included in downloadable reports, stored in the device, or transmitted to other devices.

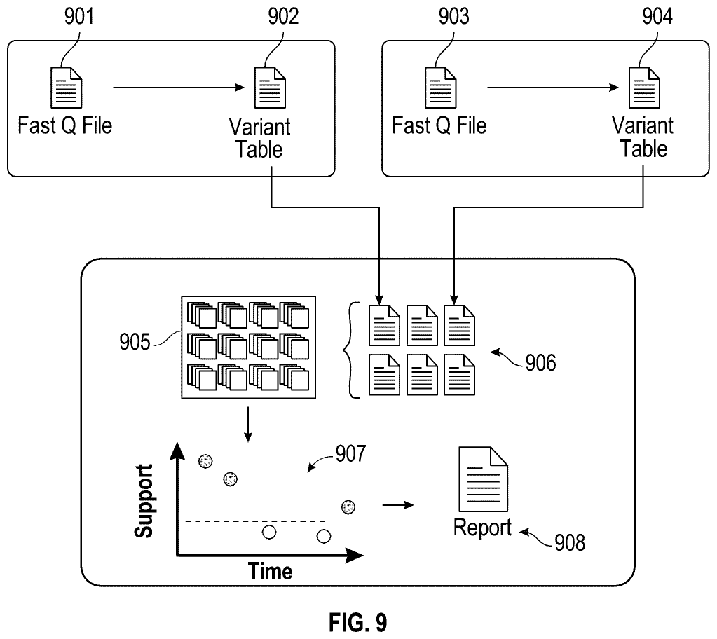

As illustrated in the schematic representation of the dynamic computer interface of FIG. 9, the sequencing reads may be obtained for each time point, stored as fastq files 901, 903, and are processed to produce modified full-variant tables 902, 904. The full-variant tables 906, which include the full-variant tables 902 and 904 alongside others that might be considered are combined into a dataset 905. The combined dataset 905 is then loaded into a user interface. The user selects markers and metrics to track and can export the results 907 into a report 908.

A software component may be presented to support the analyses of sets of the NGS data obtained at multiple time points and/or settings for the same subject, suffering from, or suspected to suffer from cancer or another condition. The device may enable a user with multiple datasets for a given subject to visualize them jointly, across multiple samples.

In one embodiment, data may be obtained at multiple time points for the same subject using the same NGS assay. In one embodiment, a first dataset may have been obtained using a first NGS assay and a second dataset may then have been obtained for the same subject at different time points using a second NGS assay.

In one embodiment, data may have been obtained at multiple time points for the same subject with a variety of NGS assays.

FIG. 10 illustrates an embodiment of the workflow of the device 1001. As illustrated in the schematic representation of the software device 1001 of FIG. 10, variant tables (VT) 1002-1005 can be uploaded via APIs for automated uploads 1017 from a genomic platform (in grey). Once uploaded into the system, variant tables (VT) 1006-1007, 1009-1010 are combined to produce a new combined dataset (combined VT) 1011, potentially written as a file. The combined VT 1011 is consumed by the dynamic user interface that allows the user to select markers (“Mar 1” to “Mar 5”) 1012 and parameters (“Par 1” to “Par 5”) 1013 for the displays 1015. The user has the capacity to export the results into a report 1016.

In some embodiments, the device 1001 is integrated in a genomic platform. However, in other embodiments, the device 1001 is a stand-alone program.

In an embodiment, the device may receive a variant table (VT) 1002-1005 for each sample as an input. The VT 1002-1005 may be in any suitable format, for example a vcf-format file. However, the device 1001 may be agnostic to the input format. In some embodiments, the uploaded VT 1006-1010 list the variants detected in the corresponding sample, with associated support. In some embodiments, the VT 1006-1010 list all positions with an alternative allele supported in the corresponding sample by at least one sequencing read, with their associated support values. The support values may include the number of reads, number of duplex sequences, and/or number of groups of reads potentially originating from the same molecule. In some embodiment, an annotation of the variants, such as predicted pathogenicity, coding consequences, frequency in populations, and the like, is provided.

In some embodiments, the device may include a user interface for the user to upload the VT 1002-1005 files and information about the VT 1002-1005 source, such as the time at which the sample was taken or the identifier of the subject. In such embodiments, variant annotation 808 can be included in the uploaded information, for instance as part of a full VT 807 in text format.

In some embodiments, the VT 1006-1010 files and annotation can be received from the genomic analysis platform to which the device is connected via an API. In such embodiments, the device includes a user interface for the user to select the uploaded VT 1006-1010 files to include in one interpretation and add and/or edit information about the samples, such as the date or identifier of the sample. In such embodiments, the annotation 808 can be passed from the genomic analysis platform as a plain-text full VT 807.

In some embodiments, the sample information, such as sampling time and patient identifier, is stored by the file management system 605 from FIG. 6. In some embodiments, the sample information, such as sampling date and patient identifier, may be reported in the VT 1006-1010 files as metadata.

The device may extract and reconcile the information stored in the different inputs. The device can then combine the information from the different samples to produce a single combined VT 809. In some embodiments, this combined VT 809 may be written as a file. In other embodiments, the combined VT 809 may be transmitted directly to the interface. In some embodiments, the samples can be obtained with different NGS assays and the positions covered by all assays are considered. In an embodiment where the sample VTs 1006-1010 include the identified variants and the associated support values, for each position detected in at least one of the samples as having a variant, the status and support values may be reported for all the samples having the variant. In an embodiment where the sample VTs 1006-1010 include all positions with at least one read supporting a reference allele, the support values for each position with an alternative allele supported by at least one read in a sample may be reported. The support value may be zero in samples where no reads supporting the alternative allele were detected. In an embodiment where annotation 808 information is provided for the detected variants or alternative alleles, the annotation 808 can be transferred to the combined VT 809.

The combined VT 809, which may comprise the information for all variants or positions with alternative alleles, can be uploaded to the user interface. The user can adjust the thresholds for variant inclusion. In conventional systems, the volume of data uploaded may pose a technical problem. However, the system as described here may accommodate large data volumes resulting from the combination of large variant tables from multiple samples, for example via the gradual upload of such data as the user interacts with the user interface.

In an embodiment, the results may be obtained using the same NGS assay, applied to multiple samples, which can represent multiple time points for one subject or different subjects. In another embodiment, the results may be obtained with different NGS assays for the different samples and may correspond to multiple time points for one subject, or to multiple subjects.

In some embodiments, the samples can be obtained with different NGS assays, where the positions covered by all assays may be considered. In an embodiment where the sample VTs include the identified variants and the associated support values, for each position detected in at least one of the samples as having a variant, the status and support values may be reported for all the samples having the variant. In an embodiment where the sample VTs include all positions with at least one read supporting a reference allele, the support values for each position with an alternative allele supported by at least one read in a sample may be reported. The support value may be zero in samples where no reads supporting the alternative allele were detected.

Independently of the source of the data, the described computer-implemented method may combine datasets obtained for multiple samples, present them in a dynamic user interface, and/or produce reports based on user-selected parametrization (e.g., for example, facilitating use for tumor-informed and tumor-uninformed scenarios). As a nonlimiting example, as illustrated in FIG. 22 the method of the present disclosure may include the following steps. Referring to step 2201, for each position at which an alternative allele was identified in at least one time point, the number of molecules, reads, and/or duplex sequences supporting the reference and alternative alleles obtained per time point are combined to produce a single dataset including the information for all positions and all time points. If available, referring to step 2202, annotation is added for each position at which an alternative allele was detected in at least one time point.

Referring to step 2203, the dataset can be loaded into a dynamic software interface. The user can select markers of interest based on criteria of their choice, which can include the support values or annotation. The user can also classify some markers as corresponding to categories, such as germline variants, clonal hematopoietic variants, or measurable residual disease variants.

Referring to step 2204, the dynamic user interface can allow the user to display support metrics for the selected markers, or markers classified in a given category, for multiple samples.

Referring to step 2205, based on a user-defined threshold, the presence or absence of each marker in each sample can be deduced from any of the number of groups of reads potentially originating from the same molecule, number of duplex reads, number of reads, and/or allele frequencies.

Referring to step 2206, the occurrence of measurable-residual disease (MRD) in a sample is detected if the number of selected markers above the selected threshold exceeds a user-defined proportion of selected markers or an absolute number of selected markers. In some embodiments, the presence or absence of each selected marker may be displayed on the dynamic interface. In some embodiments, metrics related to the abundance of each selected marker, such as the allele fraction, may be displayed on the dynamic interface. In some embodiments, metrics related to the support for each marker, such as the number of groups of reads potentially originating from the same molecule, number of duplex reads, and number of reads, may be displayed.

Referring to step 2207, a measurable-residual disease (MRD) score may be computed based on the comparison of the expected rate of errors across the selected markers and the observed support across the selected markers. In other embodiments, a measurable-residual disease (MRD) score may be computed based on the number of supported markers in each sample, using user-defined thresholds.

Referring to step 2208, the visual rendering and quantitative estimators are directly updated if the user changes the thresholds, selected markers, or metric choice.

Referring to step 2209, the device provides the option to produce reports that include metrics and displays selected by the user.

Combination of Reads from Different Aliquots

The computer-implemented method may be configured to combine the sequencing reads obtained for multiple replicates from the same sample, as illustrated in FIG. 8. As a nonlimiting example, a user can thus divide a nucleic acid sample that is above the recommended concentration into multiple aliquots, perform independent library preparation, and sequencing on each aliquot, and obtain genetic information from the sequencing reads from all aliquots.

As a nonlimiting example, without pooling of reads from different aliquots, the user would not be able to increase the amount of starting material above the recommended limit. The limit of detection would then be reduced.

### Special Variant Table

The method disclosed herein may include a bioinformatic pipeline that creates a special variant table that reports the number of groups of reads potentially originating from the same molecule (molecular count), number of reads, and/or duplex sequences, for all sites within the targeted regions with reads supporting an alternative allele.

The method thereby may differ from the state of the art, where the variant table usually includes only variants with sufficient support to be called with confidence in the analyzed sample. This special variant table is contemplated to solve two distinct problems. First, it can retain all the information and thereby allows the subsequent reporting of a low-support variant from a sample for a variant that is detected with high support in a different sample. Second, it can ensure that subsequent users can retrieve the support information for alternative alleles at any position.

As a nonlimiting example, different formats could be developed to store the information from all positions. The level of customization possible in the interface may depend on the granularity of the information retained within the variant table.

### User-Selectable Markers and Thresholds

As described in detail above, the interface may provide assay agnostic visualization of multi-point NGS data based on customizable selection of markers and thresholds. It is contemplated that this permits the user to access the input variant tables in a customizable way.

In an embodiment, because the bioinformatic pipeline retains and combines support metrics for all positions with an alternative allele in at least one sample, the user has access to the full evidence in the input variant tables, in a fully customizable way.

In various embodiments, the set of markers can be selected manually by the user, who can take into account the marker pathogenicity or protein consequences, support values, or other criteria, such as their working knowledge of the disease state and molecular pathways associated with the disease state.

As a nonlimiting example, the inference of the presence of markers and overall MRD score can rely fully on user-defined thresholds, based on the user's knowledge of the subject's disease state and the relevant markers specific to the subject's individual disease state. As a non-limiting example, the user can utilize a genomic profile obtained on a subject's tumor (e.g., after surgery) to choose which relevant markers would be optimal to utilize during the MRD assessment.

As a nonlimiting example, an alternative embodiment may consist of using pre-defined rules or thresholds for the selection of markers and the subsequent assessment. For example, the choice of markers could include all detected variants with a predicted pathogenicity or with a support value above a predefined threshold.

As another non-limiting example, an alternative embodiment may comprise setting rules or thresholds based on properties from the input VT and annotation. For example, the considered markers may include those above a given percentile of support.

### Dynamic Interface

As described in detail above, the interface may be dynamic and may respond in real time to user-selected changes in the markers, metrics, and/or thresholds. The user may therefore explore different scenarios, focusing for example on different metrics. The user may also explore the consequences of selecting different sets of markers, in a fully customizable way. The user may further explore the effect of considering more or fewer time points, together with the previous customization.

In some embodiments, the interface may report the information statically, based on markers and metrics identified with pre-defined rules and/or criteria. In an embodiment, the user could select any of the markers and/or thresholds independent of the interface and the selection may be inputted into the interface, which would then report the results statically.

It is contemplated that the user interface can provide access to multiple displays and metrics to explore and interpret the combined VT. The user interface may be interactive and the user can use buttons, or other interface means, to make and change choices of markers and parameters. The displays and metrics can respond dynamically to the user choices and may be reflected on the user interface.

### MRD Score

In an embodiment, an MRD score can be calculated for each sample based on the user-selected markers. For example, the device may calculate the MRD score jointly across multiple, user-selected markers within each sample. The score may consider the total noise and total signal across all the user-selected markers. The score may be recalculated if the user changes the selection of markers, or any other parameter described above. In some embodiments, the score may be provided as an available metric at the user interface. In some embodiments, the score may be stored and/or transmitted to other devices. In some embodiments, the MRD score may be displayed as a comprehensive report.

Unlike conventional approaches that merely report variant allele fraction (VAF) or tumor content without statistical assessment, the present disclosure provides a novel statistical MRD score that quantitatively evaluates the probability that, for example, observed support across multiple user-selected markers is due to technical noise rather than residual disease. This statistical approach differs fundamentally from binary scoring systems (positive/negative) that rely on simple thresholds. The computation of this novel MRD score is enabled by the system's ability to combine probabilities obtained for all markers to determine the likelihood that all measurements are attributable to technical noise. The dynamic user interface may allow full customization of marker selection and thresholds, enabling comprehensive reporting of support values and the calculation of this novel MRD score that considers the collective signal-to-noise ratio across all user-selected markers. The dynamic nature of the interface may enable real-time updates of the MRD score as the user adds or remove selected markers, incorporates more or fewer data sets, or changes thresholds and parameters.

For each of the user-selected markers, a measurement of the number of groups of reads potentially originating from the same molecule (hereafter ‘molecules’) supporting the presence of an MRD marker and of the total number of molecules covering the marker position may be determined. In one embodiment, a technical noise may be obtained for the user-selected markers. The technical noise may be a number of molecules that would support the presence of MRD due to technical noise, given a precomputed error rate and the total number of molecules covering the marker position.

In an embodiment, the probability that the measurement for each of the user-selected markers can be explained by the technical noise is computed, for example, using a Binomial model. In some embodiments, the p-value can be reported as the absolute value of the log, in base 10, of the probability that the measurement for one user-selected marker can be explained by technical noise. The probability that the measurements for all of the user-selected markers can be explained by technical noise can then be computed, as the product of the marker-level p-values. In some embodiments, the sum over each of the absolute values of the marker-level p-values in log base 10 can be reported as the MRD score. In other embodiments, the probability can be rescaled and reported on a different scale (e.g., between 0 to 1, 1, to 5, or 0 to 100).

As a nonlimiting example, the MRD score could be calculated differently. For instance, in one embodiment, the probability of each marker being present could be calculated based on the number of reads or groups of reads supporting the marker and the number of supported markers could be presented at the user interface. One advantage of the method and device disclosed herein is that the MRD score can consider all of the information of all user-selected markers at once and can thus be dynamically recalculated for different marker choices.

In an embodiment, the MRD score may be calculated using machine learning.

### Extension to Other Types of Genetic Data

The computer-implemented interface may be used with any NGS assay, whether based on whole-genome sequencing, hybrid-capture target enrichment, or amplicon target enrichment. The special variant table reporting the support for all reads may be created by the bioinformatic pipeline analyzing the data. In its absence, the computer-implemented interface may still be used, but the ability to access support values for variants in a sample in which they were not reported would be limited. The user interface may be used with non-NGS genetic data, for example obtained via microarrays, quantitative PCR, digital data, and the like. The user interface may also be able to mix genetic data obtained with different methods.

The tracked markers may be single nucleotide variants (SNVs), insertions/deletions (indels), copy-number variants (CNVs), fusions, other structural variants rearrangements, or other individual mutations. In some embodiments, the tracked markers are biomarkers resulting from the combination of multiple aforementioned individual markers. In some embodiments, the tracked markers are genomic signatures, such as tumor mutational burden (TMB), genomic instability, microsatellite instability, methylation patterns, gene expression profiles, or other signatures inferred from genetic data. The support metrics, reported in variant lists from individual samples, may be the metrics most adapted to the genomic signatures investigated. Similarly, the MRD score may be modified to be based on the expected noise and observed signal for the genomic signature of interest.

The system and method may be adapted for use with a sequencing-by-synthesis apparatus. The analytical pipeline and dynamic interface could however be adapted to match other sequencing approaches. The kit reagents could similarly be adapted to match other sequencing technologies.

### Dynamic Reporting of Other Metrics

The current method may include calculations and reports of different metrics for the support of variants and the overall MRD score. The interface can similarly be used to track different types of genetic variants, including as non-limiting examples single-nucleotide variants (SNVs), insertions and deletions, copy-number variants (CNVs), gene fusions, expression levels, or splice variants. An unlimited variety of support metrics and overall metrics could be used and integrated within the same framework. For instance, other MRD scores could be added as alternatives for the user to select to display. As another example, different metrics might be computed for different subsets of the genomes (e.g., per chromosome), and displayed as different variables. As a further example, metrics and displays could be segmented among variant types (e.g., structural variants versus short variants). As a further example, metrics could be added to detect changes among samples representing multiple points along a time series.

Support metrics may refer to quantitative measurements that indicate the level of evidence supporting the presence of a genetic variant in a sample. Support metrics may include, but are not limited to, the number of sequencing reads, the number of unique molecules, the number of duplex sequences, variant allele frequency (VAF), and statistical confidence scores associated with a detected variant or alternative allele.

The current system and method may include calculations and reports of different metrics for the support of variants and the overall MRD score. Other support metrics could be used and integrated within the same framework.

### Application to Diverse Genetic Datasets

As discussed in detail above, the software device described here is compatible with genetic analysis outputs obtained with a variety of NGS assays. The sequencing library can have been obtained with different methods, which can include as non-limiting examples whole-genome sequencing library, targeted enrichment using PCR-based targeting (also known as amplicon sequencing), targeted enrichment using capture probes, or other targeted enrichment methods. The genetic analysis outputs can be based on sequencing data obtained using as non-limiting examples sequencing-by-synthesis, sequencing-by-binding, nanopore sequencing, Sanger sequencing, or SNP arrays. It is contemplated that the bioinformatic pipeline to combine the genetic analysis outputs from multiple samples and the dynamic interface can be adapted to fit differences in variant report and support metrics.

### Calling of Variants Per Sample

In an embodiment, the method may identify and store the support for all alternative alleles for each sample, before merging the information for samples spanning multiple time points, settings, and/or individuals. In one embodiment, the method may include calling the variants independently for each sample, for example based on a threshold of support, and then combining the called variants across multiple time points, settings, and/or individuals.

### Static Report of Variant Progression and MRD Status

The MRD assessment interface included in the method may be dynamic, responding in real time to choices (markers, thresholds, metrics) performed by the user.

In one embodiment, the analytical pipeline may produce a calculation of a MRD score using a preset set of rules for the marker choice, threshold, and metrics.

In an embodiment, the user may be provided with the opportunity to implement their choices (markers, thresholds, metrics) before generating the overall MRD metrics and displays, for example via customizable pipeline parameters or input files.

### Monitoring of Cancer Mutations Beyond MRD

A goal of the method and device disclosed herein may be to support the detection and monitoring of somatic genetic diseases, such as cancers. In one embodiment, multiple genetic analysis outputs are produced for multiple samples. In an embodiment, the multiple samples may be collected at multiple time points, for example, corresponding to a time point before treatment and different time points after treatment.

In one example, the method and device may be utilized in the detection of measurable residual disease (MRD), however, other uses are contemplated and the method and device are not restricted to such specific cases. For example, the system, method, and device could be used to monitor blood cancers (using blood samples) or solid tumors, via the analysis of cell-free circulating DNA (cfDNA). As non-limiting examples, the monitored tumor may include lung cancer, breast cancer, kidney cancer, prostate cancer, or colorectal cancer.

Indeed, as the interface can be used to compare the frequency (with different support metrics) of mutations across samples, direct applications in the context of cancer biology include: (a) tracking the emergence and spread of de novo mutations; and (b) monitoring the response of a cancer to a treatment by tracking changes in the frequency of tumor mutations.

These applications may be performed in the presence or absence of MRD.

### Monitoring of Genetic Markers for Other Purposes

The described method and device could be used to monitor the appearance and changes in frequency of any genetic mutations. For example, one possible application could be in the monitoring of variant (allele) frequencies within populations. This could be used, for example, to monitor the appearance and resurgence of antibiotic resistance mutations in microbial populations or the appearance and spread of novel viral variants. Another application could be in the monitoring of microbial species diversity using environmental DNA, for example by tracking the emergence of known pathogens using barcoding NGS data collected at multiple time points.

### Comparison of Samples Taken at a Single Time Point

In an embodiment, the method can be used to compare samples, from a single subject, taken at different time points. In another embodiment, the method can also be used to compare different samples, for example obtained from different subjects, taken at a single time point. It is contemplated that comparing different samples taken at a single time point may be beneficial before, during, or after clinical trials, to compare the level of support for different variants across multiple individuals. This may, for example, help evaluate treatment effects. Similarly, the level of support for a given allele can be compared among individuals from a population, in the context of association studies, population genomics, or analyses of candidate genes.

Example Embodiments of Interfaces within the Disclosed Device

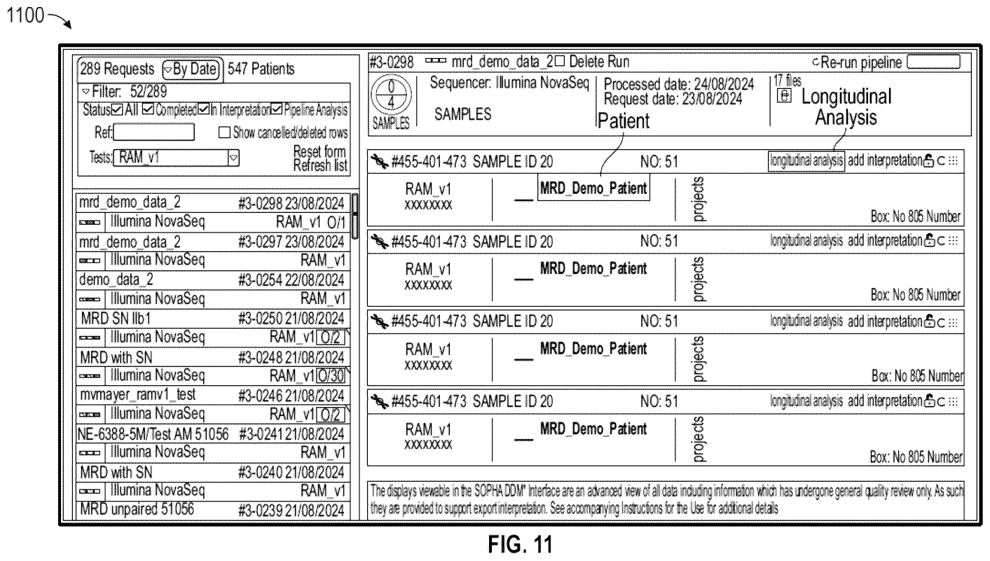

FIG. 11 illustrates access to the MRD interface 1100 from a broader genomic platform. In one embodiment, the result of one genetic analysis can be directly exported to the dynamic interface, named “longitudinal analysis and interpretation” in FIG. 11.

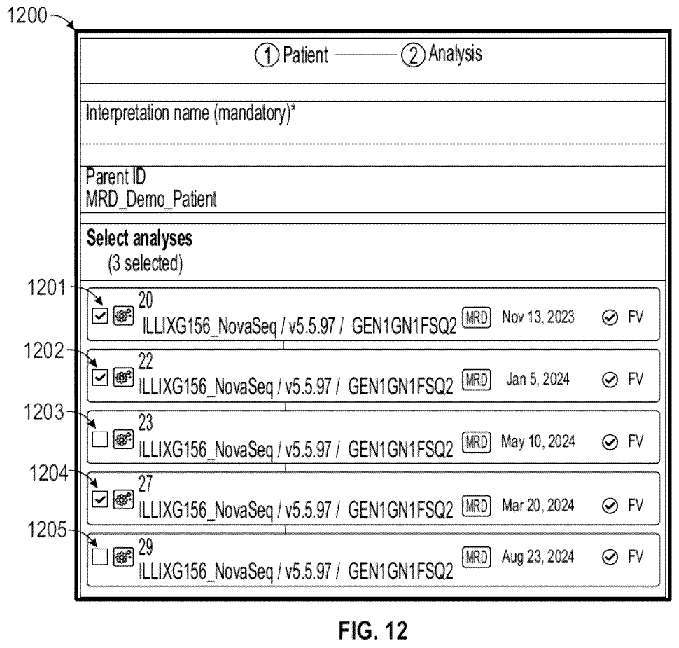

FIG. 12 illustrates genetic analysis outputs uploaded in the interface 1200, which may be selected by the user for inclusion in the multi-sample interpretation. In FIG. 12, analysis outputs “20”, “22”, and “27” 1201-1202, 1204 are selected, while analysis outputs “23” and “29” 1203, 1205 are not used.

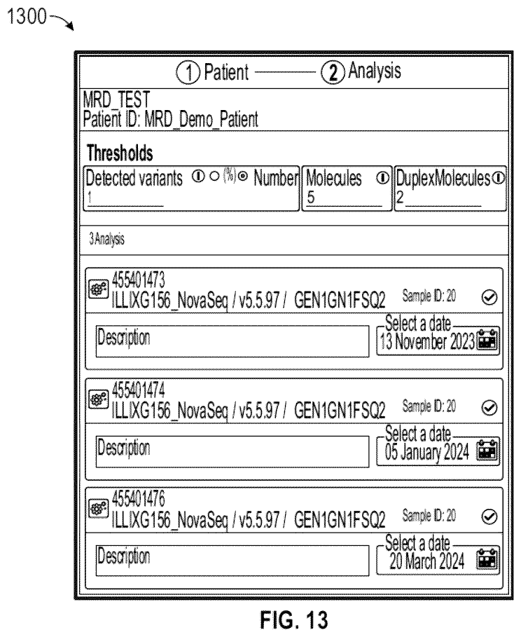

FIG. 13 illustrates an example of an interface 1300 for the user to input thresholds for the interpretation.

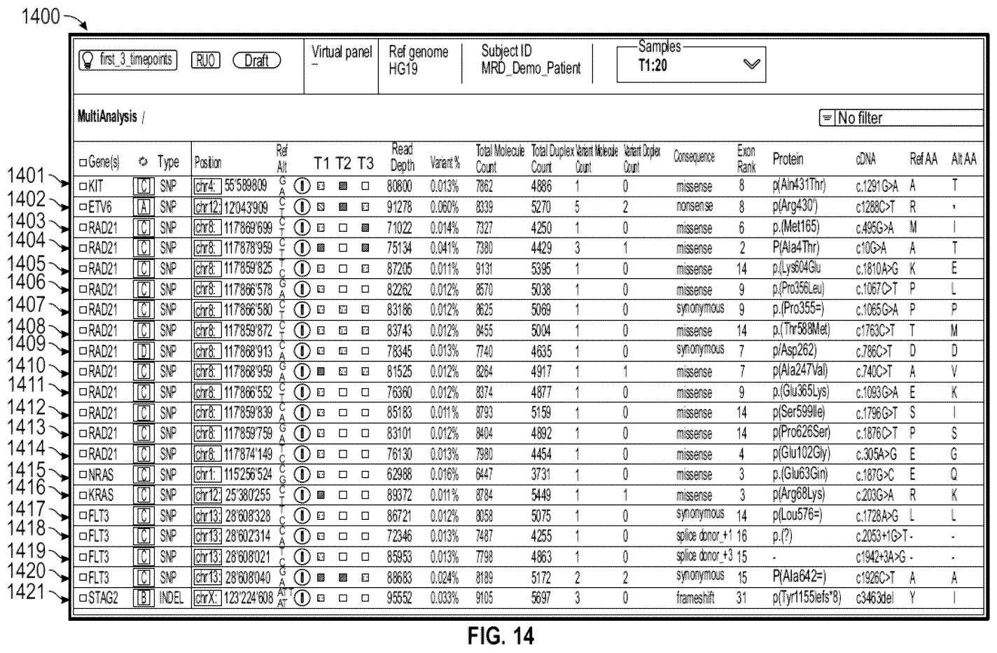

FIG. 14 illustrates an interface 1400 showing a list of variants 1401-1421 reported in at least one of the selected analyses. For each variant 1401-1421, a color-coded support may be reported for each of the selected analyses, independently for each of the time points (′T1′, ‘T2’, and ‘T3’ in this example).

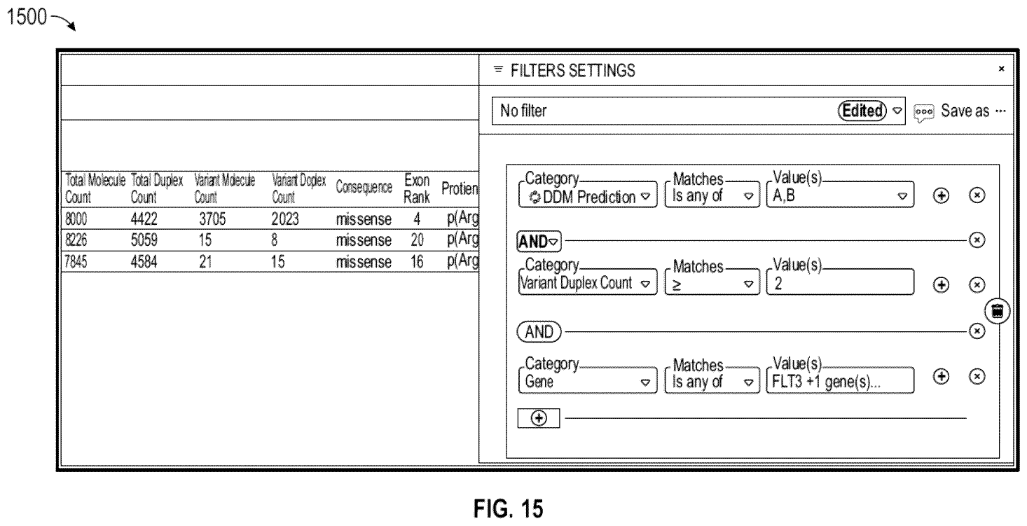

FIG. 15 illustrates an interface 1500 showing a tool for users to filter variants based on criteria of their choice.

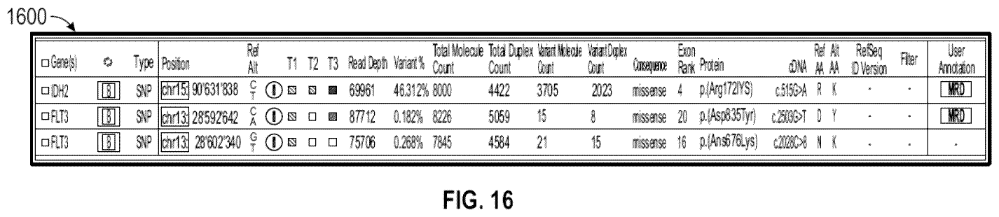

FIG. 16 illustrates a user-selected classification interface 1600 of variants as MRD markers, as visible on the right.

FIG. 17 illustrates an interface 1700 showing an example of interpretation. In such an example, the number of detected markers 1701, based on user-selected thresholds, is plotted through time. For the purposes of this example, for each sample, the MRD status 1702, based on user-selected thresholds, is indicated on the left with a minus (MRD negative), an exclamation mark 1703 (for MRD-negative based on user-selected markers, but positive based on overall MRD score), or a plus 1704 (MRD positive). A MRD score 1705 is calculated across markers and reported in the middle column. The status of each marker may be reported with colored boxes on the right. Conventional approaches include interfaces that are not dynamic, and instead require regeneration of a new plot after changing the options. In addition, such conventional options lack many of the metrics described herein (e.g., thresholds for MRD positivity, MRD score). The dynamic user interface of the present disclosure, as illustrated in FIGS. 17-19, provides real-time updates to charts and MRD scores in response to user-selected changes in considered markers, metrics, and thresholds without requiring regeneration of plots. This dynamic capability allows users to explore different scenarios by adjusting parameters through the interpretation management system 602 and immediately visualizing the impact on the combined dataset 314, enabling a level of customization not available in conventional systems. Furthermore, the MRD score calculation described herein considers the total noise and signal across all user-selected markers and is recalculated in real-time if the user changes the selection of markers or other parameters, providing immediate feedback that supports integration of clinical knowledge in the assessment.

FIG. 18 illustrates an example of an interpretation interface 1800. In such an example, the number of detected markers 1804, based on user-selected thresholds, is plotted through time 1806. For the purposes of this example, for each sample, the MRD status 1807, based on user-selected thresholds, is indicated on the left with a minus 1803 (MRD negative), an exclamation mark 1802 (for MRD-negative based on user-selected markers, but positive based on overall MRD score), or a plus 1801 (MRD positive). A MRD score 1805 is calculated across markers and reported in the middle column. The status of each marker may be reported with colored boxes on the right. The full level of evidence may be shown when placing the cursor atop a colored box.

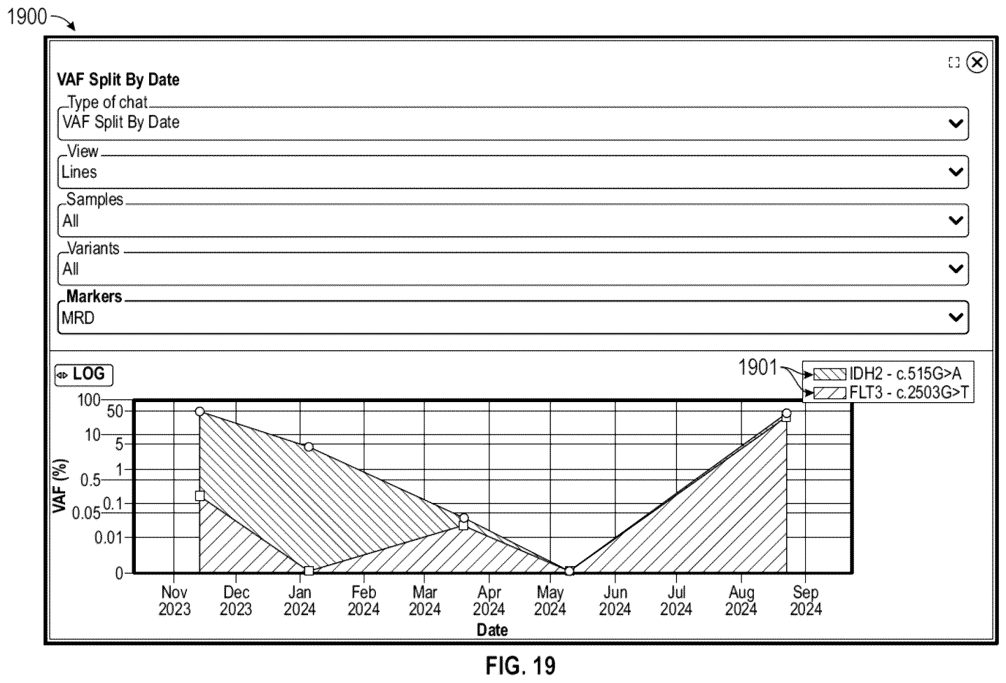

FIG. 19 illustrates an example of an interface 1900 showing a customized plot. In FIG. 19, the variant allele fraction is plotted over time for two user-selected markers 1901.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Various elements, which are described herein in the context of one or more embodiments, may be provided separately or in any suitable sub-combination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed:

1. A computer-implemented method of monitoring genetic variants of at least one or more samples from a subject, the method comprises steps of:
a. obtaining at least one or more lists of variants with support metrics, from the at least one or more sample,
b. extracting and reconciling information of the at least one or more input lists of variants and combining the information to produce a single combined list of variants with support metrics,
c. transmitting the single combined list of variants with support metrics of step (b), to a dynamic user interface,
d. selecting at least one or more markers of interest based on user selected criteria, from the dynamic user interface of step (c), and
e. creating an interpretation and displaying metrics from the set of parameters defined by the user.

2. The computer-implemented method of claim 1, wherein
(i) the input lists of variants and support metrics are selected from one or more sources, at least one or more points of time, different subjects, and/or sampling sites;
(ii) the lists of variants and support metrics are provided as a variant table in a vcf-format file; and/or
(iii) the input lists of variants lists all positions with an alternative allele supporting in the at least one or more samples by at least one or more sequencing reads, with associated support values.

3. The computer-implemented method of claim 1, wherein the monitored genetic variants comprise single nucleotide variants, insertions or deletions, copy number variants, structural variants, and/or genomic signatures inferred from more than one genomic position,
wherein the genomic signatures comprise tumor mutational burden, genomic instability, microsatellite instability, methylation patterns, and gene expression patterns.

4. The computer-implemented method of claim 1, wherein
(i) the combined list of variants lists all variants detected in the at least one or more samples, with associated support;
(ii) the associated support values comprise the number of reads, number of duplex sequences, and/or number of groups of reads potentially originating from the same molecule;
(iii) the variants are annotated and the annotations comprise predicted pathogenicity, coding consequences, known disease association, and/or frequency in population;
(iv) the variant annotation is integrated in the lists of variants; and/or
(v) the at least one list of variants is received from a genomic analysis platform via an application programming interface (API).

5. The computer-implemented method of claim 1, wherein the dynamic user interface of step (c) displays the time at which the at least one or more sample was taken, the identifier of the given subject, and/or the annotation of variants.

6. The computer-implemented method of claim 1, wherein
(i) all of the at least two input lists of variants are obtained via the same next generation sequencing (NGS) assay;
(ii) at least two of the samples are obtained via different assays and the positions covered by all assays are considered;
(iii) the lists of variants comprise identified variants and associated support values; and/or
(iv) the lists of variants comprise all positions with at least one or more reads supporting a reference allele, wherein each position with an alternative allele is supported by at least one read in one sample and the support values for the alternative alleles are reported for all samples.

7. The computer-implemented method of claim 6, wherein
(i) the NGS assay comprises a whole-genome sequencing assay, a whole-exome sequencing assay, a comprehensive profiling assay, or a disease-specific assay, wherein the disease specific assay comprises an acute myeloid leukemia (AML)-specific assay; and/or
(ii) the NGS assay is developed specifically for the subject based on a first identification of variants in said subject.

8. The computer-implemented method of claim 1, wherein the markers of interest are selected by the user based on
(i) the support values extracted from sample-specific lists of variants;
(ii) the frequency of the variant in at least one of the samples;
(iii) prior knowledge; and/or
(iv) the annotation of the variant, such as the predicted pathogenicity, coding consequences, known disease association, and/or frequency in population.

9. The computer-implemented method of claim 8, wherein the user classifies variants as germline variants, clonal hematopoietic variants, or measurable residual disease variants based on
(i) the frequency of the variant in at least one of the samples;
(ii) prior knowledge; and/or
(iii) the annotation of the variant, such as the predicted pathogenicity, coding consequences, known disease association, and/or frequency in population.

10. The computer-implemented method of claim 1, wherein at least one metric is
(i) computed based on the number, proportion and/or support of user-selected markers;
(ii) reported in the dynamic user interface, stored in the device, and/or transmitted to another device;
(iii) a measurable-residual disease (MRD) score computed based on the comparison of expected rate of errors across the selected markers and the observed support across the selected markers; and/or
(iv) measurable-residual disease (MRD) score based on the number and/or proportion of user-selected markers exceeding a user-defined threshold of support.

11. The computer-implemented method of claim 1, wherein the samples are used to investigate and/or monitor a somatic genetic disease.

12. The computer-implemented method claim 11, wherein at least one of the input lists of variants is derived from
(i) a biopsy;
(ii) a liquid biopsy; and/or
(iii) cell-free DNA (cfDNA).

13. A computer system for dynamically reporting to a user (i) clinical data of at least one or more samples, (ii) at least one or more samples databases comprising for at least each sample, a set of clinical parameters associated with clinical data for a given subject, and (iii) a set of display parameters for each diagnostic status, the computer system executing the steps of:
a. obtaining at least one or more input results as lists of variants (VT), from each sample,
b. extracting and reconciling information of the at least one or more lists of variants and combining the information to produce a single combined list of variants,
c. transmitting the single combined list of variants of step (b), to a dynamic user interface,
d. selecting at least one or more markers of interest based on user selected criteria, from the dynamic user interface of step (c), and
e. creating an interpretation and displaying support metrics from the set of parameters obtained from the dynamic user interface.

14. The computer system of claim 13, wherein
(i) the input results originate from at least one or more sources, at least one or more points of time, one or more distinct subjects, and/or distinct sampling sites;
(ii) the list of variants is received as a variant table in a vcf-format file;
(iii) the list of variants single nucleotide variants, insertions or deletions, copy number variants, and/or structural variants;
(iv) the list of variants comprises (a) the variants detected in the sample, with associated support, and/or (b) all positions with an alternative allele supporting in the sample by at least one sequencing read, with associated support values;
wherein the associated support values comprise the number of reads, number of duplex sequences, and/or number of groups of reads potentially originating from the same molecule;
(v) the variants are annotated and the annotations comprise predicted pathogenicity, coding consequences, known disease association, and/or frequency in population;
(vi) the variants are genomic signatures, such as genomic instability, tumor mutational burden, microsatellite instability, methylation profile, and/or gene expression profile; and/or
(vii) the list of variants is received from a genomic analysis platform via an application programming interface (API).

15. The computer system of claim 13, wherein the dynamic user interface of step (c) displays the time at which the at least one or more sample was taken, the identifier of the given subject, and/or variant annotation.

16. The computer system of claim 13, wherein
(i) all of the at least two input lists of variants are obtained via the same next generation sequencing (NGS) assay;
(ii) at least two of the samples are obtained via different assays and the positions covered by all assays are considered;
(iii) the lists of variants comprise identified variants and associated support values; and/or
(iv) the lists of variants comprise all positions with at least one or more reads supporting a reference allele, wherein each position with an alternative allele is supported by at least one read in one sample and the support values for the alternative alleles are reported for all samples.

17. The computer system of claim 16, wherein the NGS assay
(i) comprises a whole-genome sequencing assay, a whole-exome sequencing assay, a comprehensive profiling assay, or a disease-specific assay; and/or
(ii) is developed specifically for the subject based on a first identification of variants in said subject.

18. The computer system of claim 13, wherein the user selected criteria for the selection of markers of interest of step (d) comprises (i) the support values extracted from sample-specific VT, (ii), the frequency of the variants in at least one of the samples, (iii) prior knowledge, and/or (iv) the annotation of the variants, such as predicted pathogenicity, coding consequences, known disease association, and/or frequency in population.

19. The computer system of claim 13, wherein at least one metric
(i) is computed based on the number, proportion and/or support of user-selected markers;
(ii) is reported in the dynamic user interface, stored in the device, and/or transmitted to another device;
(iii) is a measurable-residual disease (MRD) score computed based on the comparison of expected rate of errors across the selected markers and the observed support across the selected markers; and/or
(iv) is a measurable-residual disease (MRD) score based on the number and/or proportion of user-selected markers exceeding a user-defined threshold of support.

20. A method to calculate a measurable-residual disease (MRD) score based on a selection of markers, the method comprises the steps of:
a. computing, for each marker, a level of support expected due to technical noise in an absence of the marker based on a pre-computed error rate and a total number of reads and/or number of groups of reads potentially originating from the same molecule covering a marker position;
b. measuring, for each marker, the level of support as the number of reads and/or number of groups of reads potentially originating from the same molecule supporting a presence of the marker;
c. computing, for each marker, a probability that the measured support is due to technical noise;
d. combining the probabilities obtained for all markers to obtain a probability that all measurements are due to technical noise; and
e. reporting the combined probability from step (d) as the MRD score.
